SEQUENCE LISTING (18 sequences)

SEQ ID NO: 1 - protein, 225 aa, synthetic construct
  REGION 1..225: note = C Chain C, Spike glycoprotein receptor binding domain
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFSG

SEQ ID NO: 2 - protein, 19 aa, synthetic construct
  REGION 1..19: note = Signal sequence for the spike glycoprotein, Chain A of SARS-CoV-2
MGILPSPGMPALLSLVSLL

SEQ ID NO: 3 - protein, 1253 aa, synthetic construct
  REGION 1..1253: note = Mature spike glycoprotein, Chain A of SARS-CoV-2
SVLLMGCVAETGTQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNEVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGRENLYFQGGGGSGYIPEAPRDGQAYVRKDGEWVLLSTFL

SEQ ID NO: 4 - DNA, 976 bp, synthetic construct
  misc_feature 1..976: note = Promoter+RBD
  source 1..976: mol_type = other DNA
cctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggtgaaccgaactgccttctgcctttctctgaccgccgccctgattctgaccgcctgcagcagcggagggggtggtcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttagcggctaaaggcctgcagg

SEQ ID NO: 5 - protein, 251 aa, synthetic construct
  REGION 1..251: note = RBD25 produced in NOMV
MVNRTAFCLSLTAALILTACSSGGGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFSG

SEQ ID NO: 6 - protein, 280 aa, synthetic construct
  REGION 1..280: note = RBD55 produced in NOMV
MNRTAFCLSLTAALILTACSSGGGGSGGGGVAADIGAGLADALTAPLDHKDKGLQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFSG

SEQ ID NO: 7 - protein, 251 aa, synthetic construct
  REGION 1..251: note = RBD expressed in mammalian cells
MEIKVLFALICIAVAEALERVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGHHHHHH

SEQ ID NO: 8 - DNA, 6683 bp, synthetic construct
  misc_feature 1..6683: note = pFP12 RBD plasmid
  source 1..6683: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcataaatccgttttatagccgcctgaatttgcttataggcgttatcttggcttatttctggaaactcacggacaaaatcagccaccgtaaaatcaaaaatcttttgattagatttcggatccatagtcccaatagttaaagctaaaattctgatttcatcaatactcaatcggtaattggcttcaataaggctattagcctttacaacaactaaatcatttggcataagacaacaaatttcctgtttaaaacaacaagcaaaatatacctgttgtttatatataaaacaacaagtattttcttaaaagttgtctataacaggaaatttgttgtcttataacaggaaatttgttgtcgtataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttctttttttcaggatttaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagttattttttacctttttcaatttcttcattgataaatgcactcaattcatcaaatttcttgtcatcattgataaatttacgcaacttagggaagtttctatctacatctaaaagagggttatttattatttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcaaggggcatataaagcggctcaagagcgaatggagcgagaaaaacagcaccaacaggaaaaacaacagagacatttagagcgagagagtggtttgagcttgtagctaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggtgaaccgaactgccttctgcctttctctgaccgccgccctgattctgaccgcctgcagcagcggagggggtggtcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttagcggctaaaggcctactagtcggccgtacgggccctttcgtctcgcgcgtttcggtgattaagtgcggtcatcttcggtttccgtgtttcgtaaagtctggaaacgcggaagtcccctacgtgctgctgaagttgccccaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 9 - DNA, 5185 bp, synthetic construct
  misc_feature 1..5185: note = pUC18-Lpxl1KO-FHbp25RBD-KAN plasmid
  source 1..5185: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttatgtgtatcgagatgaaatttatattttttgtactgtatgttttgcagtttctgccgtttgcgctgctgcacaagattgccgacctgacgggtttgcttgcctaccttctggtcaaaccgcgccgccgtatcggcgaaatcaatttggcaaaatgtttttccgaatggagtgaggaaaagcgtaaaaccgtgttgaaacagcatttcaaacacatggcgaaactgatgttggaatacggtttatattggtacgcgcctgccggacgtttgaaatcgctggtgcgctaccgcaataagcattatttggacgacgcgctggcggcgggggaaaaagtcatcatcctgtatccgcacttcaccgctgcagttctcgaggcagtgactaactaggaggaataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacccctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggtgaaccgaactgccttctgcctttctctgaccgccgccctgattctgaccgcctgcagcagcggagggggtggtcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttagcggctaaaggcctgcagggaataatgagtcgacaggatttcggacgcaacgattcggtttttgtggattttttcggtattcagacggcaacgattaccggattgagccgcattgccgcgcttgcaaatgcaaaagtgatacccgccattcccgtccgcgaggcagacaatacggttacattgcatttctaccctgcttggaaatcctttccgggtgaagacgcgaaagccgacgcgcagcgcatgaaccgttttatcgaagacagggtgcgcgaacatccggaacaatatttttggctgcacaagcgttttaaaacccgtccggaaggcagccccgatttttactgactacgtcagacggctctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 6404 bp, synthetic construct
  misc_feature 1..6404: note = pGEM-SiaD-GalE-FHbp25RBD-SPC plasmid
  source 1..6404: mol_type = other DNA
gggcgaattcctggacacggcagaccgataaagcccgcaccgccggtaacgaggatggtttttttgtttgcggtttgcatttaatccccatatccgttggggttgcggctgacccaacgccacgaatcttccatcatttgctgcaagccgcgtttggtttcccagccggtttgttgtttggtatgggacgggtcggcaaaggaacacgccaaatcaccggcgcggcgcggtttgatttcaaatggaatggttaaacccgaagccgcttcaaatgcgcggatgatttccaataccgaagaagcgcggccggagcctaagttcagcaaatgcgtgcctgctacattactttttgcctgcatagccgcgacatggccttctgccaaatccatcacatgaatatagtcacgcatccccgtgccgtcgggggtagggtagtcatcgccaaataccgccaattgcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggtgaaccgaactgccttctgcctttctctgaccgccgccctgattctgaccgcctgcagcagcggagggggtggtcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttagcggctaaaggtctagagacgatgccgtctgaagaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctatgccgtctgaatactcgagcggaacccgggtagtggcctgtaaaagacagcatatagagatgagcaggctgtataatattaaggatttttttgtaacttctataaatataaagtaattttttaggagttatattattagggcttctaggaagctcaaatagataaatagattcaaatagattcttgttagctgattgatgaactaacttaggcatttttaagtttttagaagtatataaaattactagtaaattattggttaatttttgtattttaattaggctttggacttggttaagctgacctaaattagatatgacaaataaattgttacgtgggggggtaagataaaatggagatgttgtcaaccacattgaatcttgaaaaaactttttaggctgaaaaagagctttttttattttctttagcattattgtatctcttaaaaattaatgagaattagctatatgtaatagccaatcctctgttaataaagtaactaagttaataagcattattcaatatcagtttttttgatttgagcacctttgcgaatattgcaagcagcgaccttaccaaataatgtttcatattcgttgacgctgaagtctccattgcctgggcgtttaacccataggttatctaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 11 - DNA, 6146 bp, synthetic construct
  misc_feature 1..6146: note = pBS-FHbpKO-FHbp25RBD-ERM plasmid
  source 1..6146: mol_type = other DNA
gcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggtgaaccgaactgccttctgcctttctctgaccgccgccctgattctgaccgcctgcagcagcggagggggtggtcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttagcggctaaaggcctgcagggccaagcaaaaccattgtgaaaatgccgtccgaacacgataatttaccgttcggacggcattttgtattgcaccgtccgacggcatgcccaaggggggaaatccctattttcaggccaaccgctatataatgccgtctgaaccaacgagagaatgccatgcaagctgattttaaccgtcccgtcctggccgtcgataccggtacttcccgtttgtcgctcgcgctgcgtgccgacggcgaaacccgtctgttccatcaggaagtcggcagccgccagtccgaactgattctgccggaaatccgcaccctattccgcgatgcaggcattaccgccgccgatttgggtgcggtcgtgtacgcacagggtcccggcgcgtttaccggactgcgtatcggcatcggtgtagctcagggtttggcaacgccgtttgatacccccttaatcggcgtaccctcgctcgatgccgccgcctcgctgccgccgccgcaaagctgcatccttgccgctacggacgctcgtatgggcgaagtgttttatgcatggttcgatacgctgctcgagggggggcccggtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaccagccacggcgcatacaaattcacccgtccgcccacaggcgatgtattacgtatcgaccgcatcaaagaaatccaccaagccctgcccaatacacacatcgtgatgcacggctccagctccgttccgcaagaatggctgaaagtcatcaacgaatacggcggcaatatcggcgaaacctacggcgtgccggttgaagaaatcgtcgaaggcatcaaacacggcgtgcgcaaagtcaacatcgataccgacttgcgccttgcttctaccggcgcggtacgccgctaccttgccgaaaatccgtccgactttgacccgcgcaaatacctgagcaaaaccattgaggccatgaagcaaatctgcctcgaccgttatcttgcgtttggctgcgaaggtcaggcaggcaaaatcaaacctgtttcgttggaaaaaatggcaagccgttatgccaagggcgaattgaaccaaatcgtcaaataacaggttgcctgtaaacaaaatgccgtcccatgggcaaacttaagagtgtgttgatagtgcagtatcttaaaattttgtgtataataggaattgaagttaaattagatgctaaaaatttgtaattaagaaggagggattcgtcatgttggtattccaaatgcgtaatgtagataaaacatctactgttttgaaacagactaaaaacagtgattacgcagataaataaatacgttagattaattcctaccagtgactaatcttatgactttttaaacagataactaaaattacaaacaaatcgtttaacttctgtatttatttacagatgtaatcacttcaggagtaattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaacattcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccattacttatgagcaagtattgtctatttttaatagttatctattatttaacgggaggaaataattctatgagtcgcttttttaaatttggaaagttacacgttactaaagggaatggagataaattattagatatactactgacagcttccaagaagctaaagaggtccctttcgacggccccgggcctcagc

SEQ ID NO: 12 - DNA, 5275 bp, synthetic construct
  misc_feature 1..5275: note = pUC18-Lpxl1KO-FHbp55RBD-KAN plasmid
  source 1..5275: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttatgtgtatcgagatgaaatttatattttttgtactgtatgttttgcagtttctgccgtttgcgctgctgcacaagattgccgacctgacgggtttgcttgcctaccttctggtcaaaccgcgccgccgtatcggcgaaatcaatttggcaaaatgtttttccgaatggagtgaggaaaagcgtaaaaccgtgttgaaacagcatttcaaacacatggcgaaactgatgttggaatacggtttatattggtacgcgcctgccggacgtttgaaatcgctggtgcgctaccgcaataagcattatttggacgacgcgctggcggcgggggaaaaagtcatcatcctgtatccgcacttcaccgctgcagttctcgaggcagtgactaactaggaggaataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacccctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggtgaaccgaactgccttctgcctttctctgaccgccgccctgattctgaccgcctgcagtagcggaggcggcggaagcggaggcggcggtgtcgccgccgacatcggtgcggggcttgctgatgcactaaccgcaccgctcgaccataaagacaaaggtttgcagcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttagcggctaaaggcctgcagggaataatgagtcgacaggatttcggacgcaacgattcggtttttgtggattttttcggtattcagacggcaacgattaccggattgagccgcattgccgcgcttgcaaatgcaaaagtgatacccgccattcccgtccgcgaggcagacaatacggttacattgcatttctaccctgcttggaaatcctttccgggtgaagacgcgaaagccgacgcgcagcgcatgaaccgttttatcgaagacagggtgcgcgaacatccggaacaatatttttggctgcacaagcgttttaaaacccgtccggaaggcagccccgatttttactgactacgtcagacggctctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 13 - DNA, 6494 bp, synthetic construct
  misc_feature 1..6494: note = pGEM-SiaD-GalE-FHbp55RBD-SPC plasmid
  source 1..6494: mol_type = other DNA
gggcgaattcctggacacggcagaccgataaagcccgcaccgccggtaacgaggatggtttttttgtttgcggtttgcatttaatccccatatccgttggggttgcggctgacccaacgccacgaatcttccatcatttgctgcaagccgcgtttggtttcccagccggtttgttgtttggtatgggacgggtcggcaaaggaacacgccaaatcaccggcgcggcgcggtttgatttcaaatggaatggttaaacccgaagccgcttcaaatgcgcggatgatttccaataccgaagaagcgcggccggagcctaagttcagcaaatgcgtgcctgctacattactttttgcctgcatagccgcgacatggccttctgccaaatccatcacatgaatatagtcacgcatccccgtgccgtcgggggtagggtagtcatcgccaaataccgccaattgcctcagcgcatgcatccgggctctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatggtgaaccgaactgccttctgcctttctctgaccgccgccctgattctgaccgcctgcagtagcggaggcggcggaagcggaggcggcggtgtcgccgccgacatcggtgcggggcttgctgatgcactaaccgcaccgctcgaccataaagacaaaggtttgcagcgcgtgcagccgaccgaaagcattgtgcgctttccgaacattaccaacctgtgcccgtttggcgaagtgtttaacgcgacccgctttgcgagcgtgtatgcgtggaaccgcaaacgcattagcaactgcgtggcggattatagcgtgctgtataacagcgcgagctttagcacctttaaatgctatggcgtgagcccgaccaaactgaacgatctgtgctttaccaacgtgtatgcggatagctttgtgattcgcggcgatgaagtgcgccagattgcgccgggccagaccggcaaaattgcggattataactataaactgccggatgattttaccggctgcgtgattgcgtggaacagcaacaacctggatagcaaagtgggcggcaactataactatctgtatcgcctgtttcgcaaaagcaacctgaaaccgtttgaacgcgatattagcaccgaaatttatcaggcgggcagcaccccgtgcaacggcgtggaaggctttaactgctattttccgctgcagagctatggctttcagccgaccaacggcgtgggctatcagccgtatcgcgtggtggtgctgagctttgaactgctgcatgcgccggcgaccgtgtgcggcccgaaaaaaagcaccaacctggtgaaaaacaaatgcgtgaactttagcggctaaaggtctagagacgatgccgtctgaagaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctatgccgtctgaatactcgagcggaacccgggtagtggcctgtaaaagacagcatatagagatgagcaggctgtataatattaaggatttttttgtaacttctataaatataaagtaattttttaggagttatattattagggcttctaggaagctcaaatagataaatagattcaaatagattcttgttagctgattgatgaactaacttaggcatttttaagtttttagaagtatataaaattactagtaaattattggttaatttttgtattttaattaggctttggacttggttaagctgacctaaattagatatgacaaataaattgttacgtgggggggtaagataaaatggagatgttgtcaaccacattgaatcttgaaaaaactttttaggctgaaaaagagctttttttattttctttagcattattgtatctcttaaaaattaatgagaattagctatatgtaatagccaatcctctgttaataaagtaactaagttaataagcattattcaatatcagtttttttgatttgagcacctttgcgaatattgcaagcagcgaccttaccaaataatgtttcatattcgttgacgctgaagtctccattgcctgggcgtttaacccataggttatctaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 14 - DNA, 8275 bp, synthetic construct
  misc_feature 1..8275: note = pBS-FHbpKO-FHbp55RBD-ERM plasmid
  source 1..8275: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttggatccttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcgtaaatccgttttatagccgcctgaatttgcttataggcgttatcttggcttatttctggaaactcacggacaaaatcagccaccgtaaaatcaaaaatcttttgattagatttcggatccatagtcccgatagttaaagctaaaattctgatttcatcaatactcaatcggtaattggcttcaataaggctattagcctttacaacaactaaatcatttggcataatgccgtctgaaagacaacaaatttcctgtttaaaacaacaagcaaaatatacctgttgtttatatataaaacaacaagtattttcttaaaagttgtctataacaggaaatttgttgtcgtataacaggaaatttgttgtcttataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttcttttttcaggattaaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagatgccgtctgaacccgggatgccgtctgaaactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatgccgtctgaactcgagatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcgaggggcatataaagcggctcaagagcgaatggagcgagaaaaacagcaccaacaggaaaaacaacagagacatttagagcgagagagtggtttgagcttgtagatgccgtctgaactaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgggaaacagctatgaccatgattacgccaagcttcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgctctatcgaaggacctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgcagttacagatccaagctgtgaccggcgcctacctgagatcaccggtcaccatggagatcaaggtgctgtttgccctcatctgtattgctgttgctgaggcactcgagcgggtccaacccaccgaaagcattgtgcggttcccaaatatcaccaatctgtgtccctttggcgaagtgttcaatgctacaaggtttgcttctgtgtacgcatggaataggaaacgcatctccaattgtgtcgctgattactccgtgctgtacaattccgcctctttctcaaccttcaagtgttatggcgtttcacctaccaaacttaacgacctgtgcttcactaatgtgtatgccgactcttttgtgatacgaggcgatgaagtgagacagattgcaccagggcagaccggcaaaattgccgactacaactacaagcttccagatgactttaccggatgtgttattgcatggaactcaaacaatctggattccaaggtgggtggcaactataactacctgtatagactgttcaggaaatccaacctgaaaccattcgagcgagatataagcacagaaatctaccaggctggaagtacgccctgcaacggcgtggaagggttcaactgctacttcccattgcagagttacggattccagcctacaaacggggtgggttaccaaccctatcgtgtcgtagtcctgagttttgagctcctccatgccccagccacagtctgtggccccaagaaaagcaccaatctggtgaagaacaaatgcgtgaactttggatccggccatcatcatcaccatcactaaagctagctggccagacatgataagatacattgatgagtttggacaaaccacaactagaatgcagtgaaaaaaatgctttatttgtgaaatttgtgatgctattgctttatttgtaaccattataagctgcaataaacaagttaggcctgcaggactagtcggccgtacctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgggccctttcgtctcgcgcgtttcggtgatagcttaagtgcggtcatcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 15 - DNA, 6838 bp, synthetic construct
  misc_feature 1..6838: note = pFP12 SV40 RBD-2 plasmid
  source 1..6838: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttggatccttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcgtaaatccgttttatagccgcctgaatttgcttataggcgttatcttggcttatttctggaaactcacggacaaaatcagccaccgtaaaatcaaaaatcttttgattagatttcggatccatagtcccgatagttaaagctaaaattctgatttcatcaatactcaatcggtaattggcttcaataaggctattagcctttacaacaactaaatcatttggcataatgccgtctgaaagacaacaaatttcctgtttaaaacaacaagcaaaatatacctgttgtttatatataaaacaacaagtattttcttaaaagttgtctataacaggaaatttgttgtcgtataacaggaaatttgttgtcttataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttcttttttcaggattaaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagatgccgtctgaacccgggatgccgtctgaaactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatgccgtctgaactcgagatgccgtctgaactaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgggaaacagctatgaccatgattacgccaagcttcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgctctatcgaaggacctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgcagttacagatccaagctgtgaccggcgcctacctgagatcaccggtcaccatggagatcaaggtgctgtttgccctcatctgtattgctgttgctgaggcactcgagcgggtccaacccaccgaaagcattgtgcggttcccaaatatcaccaatctgtgtccctttggcgaagtgttcaatgctacaaggtttgcttctgtgtacgcatggaataggaaacgcatctccaattgtgtcgctgattactccgtgctgtacaattccgcctctttctcaaccttcaagtgttatggcgtttcacctaccaaacttaacgacctgtgcttcactaatgtgtatgccgactcttttgtgatacgaggcgatgaagtgagacagattgcaccagggcagaccggcaaaattgccgactacaactacaagcttccagatgactttaccggatgtgttattgcatggaactcaaacaatctggattccaaggtgggtggcaactataactacctgtatagactgttcaggaaatccaacctgaaaccattcgagcgagatataagcacagaaatctaccaggctggaagtacgccctgcaacggcgtggaagggttcaactgctacttcccattgcagagttacggattccagcctacaaacggggtgggttaccaaccctatcgtgtcgtagtcctgagttttgagctcctccatgccccagccacagtctgtggccccaagaaaagcaccaatctggtgaagaacaaatgcgtgaactttggatccggccatcatcatcaccatcactaaagctagctggccagacatgataagatacattgatgagtttggacaaaccacaactagaatgcagtgaaaaaaatgctttatttgtgaaatttgtgatgctattgctttatttgtaaccattataagctgcaataaacaagttaggcctgcaggactagtcggccgtacctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgggccctttcgtctcgcgcgtttcggtgatagcttaagtgcggtcatcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 16 - DNA, 8578 bp, synthetic construct
  misc_feature 1..8578: note = pFP12 SV-40 RBD-2 complete plasmid
  source 1..8578: mol_type = other DNA
ctgcaggccgtctgaaccgctctaaccgctttttctcggcttaatttttctgtctctgttataaaattgctattcatcttgttcttcttcaaaaaaaagttaagtaaaatacctacctaaatttggatccttactagttcgcaatctacgagcttataacctcgttttttcaattcatttaaaaaatcagattttgagcctaattttgatctattgctatcgttacccgctagaaatacccagtaattacgcaaatcttcattggtaactttcgtaatatcggtgtaatgatcttcgagtatttttaagcaatctctagcccataaaccgtactcgtgattgctcatcttagggttttgcttatcgagtttgacgaacttcccatacttgtttttatgtggaaatactggccgttttgcaacttcttcaattttttgagctgttcgttttttactaccaatcacaaaatttaaagagtgaatagtacgcccacgcttgatttgttcaacctcaacgactaaatcagatttctcgttaatctcagttattgcaggttccaaaacacgttgatttaatgaattaaatctagggtatttattttcaacctgaagccattcttttagtttttctactgtaatttcacgactaccaacagagcgatattgtgtaattagctcataaattcgaattgaatgtacactgttgaaataagcgatatgtttgagttgatattgcgtgaattgccctttaagttgcgttaggtatggcataacttcatcagtcattgcaattctaaaacgcccctctttcttgaaatatgttctagaggaaacccaacgaaattcagttacacggtctttatcttcagttttaacacttcggtcgtaaatccgttttatagccgcctgaatttgcttataggcgttatcttggcttatttctggaaactcacggacaaaatcagccaccgtaaaatcaaaaatcttttgattagatttcggatccatagtcccgatagttaaagctaaaattctgatttcatcaatactcaatcggtaattggcttcaataaggctattagcctttacaacaactaaatcatttggcataatgccgtctgaaagacaacaaatttcctgtttaaaacaacaagcaaaatatacctgttgtttatatataaaacaacaagtattttcttaaaagttgtctataacaggaaatttgttgtcgtataacaggaaatttgttgtcttataacaggaaatttgttgtcgtataacaggaaatttgttgtcgtataagtttgtaacttattgattttactggttttaaaaacgccgaaaacaagtaaaaaacaaaaatataaaaatatagggactttcgtcccttttttgggctttcagccctaattttttcttttttcaggattaaaaattacaaaacccttacagagcaagtaaacttgtttgcttgttctgcaagggttcagcaaccgtagccgtcaggcgtagggcggtagcctataaaagccatttaattttatctttaaacttccttttaaatgctttgagtgggtgtcttttatcgtactcatcaatccttttttgcattctttcgtttgctttgtgatcggcaaattttgaataagatttttccatctcatctaacattctatcaatccgttttttatgttgccatttcaggtaaacataaacacttatagcaattaaagacaatatcaatacattgtaaaaaatgattgttacaatttcgctcacagatgccgtctgaacccgggttattttttacctttttcaatttcttcattgataaatgcactcaattcatcaaatttcttgtcatcattgataaatttacgcaacttagggaagtttctatctacatctaaaagagggctatttattacttcatttagccaaaaagcccctaataaaaccttgtaatgcgtagctttcttacgcttttctgcttgttcttttgacttaatcgcacgaattttcgctttgatttcgtcctgcttgcgttgtaaatctgcttgttgctgttccaatcttgtaagtttttcgcttgccatcccgggatgccgtctgaaactagcccctttatatagttagaaattatcgttattttattcagtaggtgctaggcttgcaagtgttctgttcattacgttaaaataacgtaatgcccacttatcagtttctcttcgagaaactggtgggcaagcgtaccgcttgaccgtttcgcaatactcaacactatgccgtctgaactcgagatggcaatctatcatttaaacgttcgctattgcagtaaaagcaaagggcaatcagctcaagccaaaaacgactacatcaaccgcaatgataaatattcaaagcggttagatgatttacagttttcaggctatggtaatatgccaaaatttgccgaagataatccgcaagaattttggcgattgtcagatatttacgagcgagctaatgcccgagtttgtactgaaattgaatttgctttacctagagaattaaccctagaacaacagcaaaaattagtaagttcgtttatagaaaatacggttgatagcggtagcaataaactaccctactctttcgctatccataccgataaaaataatcataatccccattgtcatttgatattttcagaacgccaacttgacggcatagaccgtacagccgagcagttttttaaacgtgctaatactaaatccccagaaaagggcggagcgatgaaaacggcagattttcgagatcgtgagtttatccaatctgtccgaaaaacgtggagagagcaagctaatcaagccttagagcaatacggatatgccgcacgaattgacgaacgtagctacaaggaacaaggcatagagcaagccccaagagcaagaattgacagggtaacgtggcaagaattgaaccgattagagcaagaagaacgccaaatcgtgcaagagcttgcacttaaaggacaagaaattaacaaagaaaaatcctacttgcagaaaatcgaagaaaaacaggctcaaggaatgggcaaatatgaatccaaattcgcagctgcgttttctaaattatcggaaagtgccctaaaacacgatttaagcaacgaaaaagaaaaagacagtaaaatacacactcaagaagaaaaagtgcctcaaaatcgcattcaggggctttctcaagcagattttgatcagtttttaattgatgaatggctacctcaaatagaaaaatacgttaaagcccaagaaaagcgggacggaatggaagtagagatcacgcaatacgacaaggatttacagcgtattcagggagactataacaagctcacagataaaaatcagggttttctcggtttatgggaaactaaagagcaaaaagcaaagaaaaaagagcttgaagatgaatacaaacatacagcagagcaacggaacgctaaaagccaagaattagccgagtatagccaaaaaataaaagcatacgaacagaaaacgctagagccaatcaacgagaagattgccaaatatcaagctgacaaccctgaaataaaaatgcggagcttaggatttgtgaaaaaaattaaggctcgaggggcatataaagcggctcaagagcgaatggagcgagaaaaacagcaccaacaggaaaaacaacagagacatttagagcgagagagtggtttgagcttgtagatgccgtctgaactaacgccctacgcctacggcttcggttgttcaacccttaaagaactcgcaacaagttgcaaattctttaagggttcgcaataaaaacaaccgctaaacatttctgcccagcggttgaaaatttacctattcaccattacaatgatcaagcaggaaatttttttgattgccgtaaatgtccgtatatctagttgaggcacaacccgccaaagtcattgccccaaccagaacggcgaaagctaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacgggaaacagctatgaccatgattacgccaagcttcctcagcgcatgcatccgggcagcagcgcatcggctcgcacgaggtctgcgcttgaattgtgttgtagaaacacaacgtttttgaaaaaataagctattgttttatatcaaaatataatcatttttaaaataaaggttgcggcatttatcagatatttgttctgaaatgaagacgtatcgggtgtttgcccgatgtttttaggtttttatcaaatttacaaaaggaagcccatatgctctatcgaaggacctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgcagttacagatccaagctgtgaccggcgcctacctgagatcaccggtcaccatggagatcaaggtgctgtttgccctcatctgtattgctgttgctgaggcactcgagcgggtccaacccaccgaaagcattgtgcggttcccaaatatcaccaatctgtgtccctttggcgaagtgttcaatgctacaaggtttgcttctgtgtacgcatggaataggaaacgcatctccaattgtgtcgctgattactccgtgctgtacaattccgcctctttctcaaccttcaagtgttatggcgtttcacctaccaaacttaacgacctgtgcttcactaatgtgtatgccgactcttttgtgatacgaggcgatgaagtgagacagattgcaccagggcagaccggcaaaattgccgactacaactacaagcttccagatgactttaccggatgtgttattgcatggaactcaaacaatctggattccaaggtgggtggcaactataactacctgtatagactgttcaggaaatccaacctgaaaccattcgagcgagatataagcacagaaatctaccaggctggaagtacgccctgcaacggcgtggaagggttcaactgctacttcccattgcagagttacggattccagcctacaaacggggtgggttaccaaccctatcgtgtcgtagtcctgagttttgagctcctccatgccccagccacagtctgtggccccaagaaaagcaccaatctggtgaagaacaaatgcgtgaactttggatccggccatcatcatcaccatcactaaagctagctggccagacatgataagatacattgatgagtttggacaaaccacaactagaatgcagtgaaaaaaatgctttatttgtgaaatttgtgatgctattgctttatttgtaaccattataagctgcaataaacaagttaggcctgcaggactagtcggccgtacctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgggccctttcgtctcgcgcgtttcggtgatagcttaagtgcggtcatcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaaca

SEQ ID NO: 17 - DNA, 8275 bp, synthetic construct; identical to SEQ ID NO: 14
  misc_feature 1..8275: note = pFP12 SV-40 RBD-2 mobC plasmid
  source 1..8275: mol_type = other DNA

SEQ ID NO: 18 - protein, 259 aa, synthetic construct
  REGION 1..259: note = FHbp ID9
CSSGGGGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTALQTEQVQDSEDSGKMVAKRQFRIGDIAGEHTSFDKLPKGGSATYRGTAFGSDDAGGKLTYTIDFAVKQGHGKIEHLKSPELNVDLAAAYIKPDKKRHAVISGSVLYNQDEKGSYSLGIFGGQAQEVAGSAEVETANGIHHIGLAAK

(19) United States
(12) Patent Application Publication
(10) Pub. No.: US 2023/0226174 A1
(43) Pub. Date: Jul. 20, 2023

(54) SARS-COV-2 RECEPTOR BINDING DOMAIN IN NATIVE OUTER MEMBRANE VESICLES

(71) Applicant: Omvax, Inc., San Francisco, CA (US)

(72) Inventors: Gregory MOE, Oakland, CA (US); Serena GIUNTINI, Walnut Creek, CA (US)

(21) Appl. No.: 18/160,835

(22) Filed: Jan. 27, 2023

Related U.S. Application Data
(63) Continuation of application No. PCT/US2021/044012, filed on Jul. 30, 2021.
(60) Provisional application No. 63/059,031, filed on Jul. 30, 2020.

(51) Int. Cl. A61K 39/215; A61P 31/14

(52) U.S. Cl. CPC A61K 39/215; A61P 31/14; A61K 2039/521

(57) ABSTRACT

The disclosure provides native outer membrane vesicle (NOMV) vaccines containing a coronavirus receptor binding domain (RBD) modified to be a lipoprotein. Also provided are compositions comprising a meningococcal strain having a plasmid-borne gene encoding the SARS-CoV-2 RBD modified to be a lipoprotein. Also provided are a meningococcal strain and a NOMV vaccine containing a plasmid coding for the SARS-CoV-2 RBD with a promoter/enhancer and polyA sequence that provide for expression of the RBD in mammalian cells.

### CROSS REFERENCE TO RELATED APPLICATIONS

This application is a bypass continuation of International Application No. PCT/US2021/044012, filed Jul. 30, 2021, which claims the benefit of U.S. Provisional Patent Application No. 63/059,031, filed Jul. 30, 2020, the disclosures of each are incorporated by reference herein in their entireties.

### STATEMENT REGARDING FEDERALLY SPONSORED RESEARCH

This invention was made with government support under grant numbers R01AI046464 and R44AI124759 awarded by the National Institutes of Health. The government has certain rights in the invention.

### INCORPORATION OF SEQUENCE LISTING

The sequence listing that is contained in the file named “OMV0001-201BC-US,” which is 93 kilobytes as measured in Microsoft Windows operating system and was created on Jan. 27, 2023, is filed electronically herewith and incorporated herein by reference.

### BACKGROUND

SARS-CoV-2 is a coronavirus that is the cause of COVID-19 disease in humans The virus enters airway epithelial cells by binding to glycans on ciliated cells. The protein mediating binding is the S (spike) protein. A domain within the S-protein (receptor binding domain or RBD) binds to the ACE2 receptor and glycans. The structure of the S-protein alone and in complex to the ACE2 receptor has been determined. In addition, structures of the RBD in complex with neutralizing monoclonal antibodies have been determined.

Native outer membrane vesicles (NOMV) are blebbed naturally from Neisseria meningitidis (Nm) bacteria. Previously, vaccine strains have been genetically modified to (a) overexpress Factor H binding protein (FHbp), which is normally present in low abundance, (b) express mutant FHbp with low binding to host Factor H to increase antibody responses that block the interactions causing FH binding, and (c) have attenuated endotoxin, enabling use of NOMV without the detergent treatment that is normally used to decrease reactogenicity, but also results in removal or alteration of potentially protective antigens. The NOMV-FHbp with penta-acylated lipooligosaccharide (LOS) resulting from knocking out LpxL1 decreases cytokine responses in human peripheral blood mononuclear cells (PBMC), which were similar to or lower than those elicited by detergent extracted OMV vaccines that had been safely administered to tens of thousands of human subjects. To further enhance the safety of the NOMV-FHbp vaccine, the strains used to prepare the vaccine incorporate additional genetic deletions that eliminate expression of other undesirable antigens, including the group B capsular polysaccharide, and derivatives of LOS, which are known to cross-react with human glycans having similar structures.

The immunogenicity of antigens presented in NOMV is greatly increased versus comparable amounts of the recombinant protein alone. However, the most effective antibody responses require a threshold level of expression that has been achieved by using promoters engineered to produce high rates of transcription, inserting multiple copies in the bacterial genome, and transformation with a multi-copy plasmid.

Meningococcal outer membrane vesicles that contain hexa-acylated lipooligosaccharide produce inflammatory responses. Reactogenicity can be reduced by detergent extraction, however detergent treatments can result in loss of lipoprotein antigens and alterations in protein structure. The Nm strain used to produce NOMV as described herein has the lpxL1 locus disrupted, resulting in production of penta-versus hexa-acylated LOS, which results in attenuated endotoxin activity.

The NOMV platform also has adjuvant properties that enhance antibody responses. Overall, NOMV-based vaccines elicit higher titers of antibodies with broader reactivity than the corresponding recombinant proteins and may be more tolerable, since less protein may be required to provide an effective protective antibody response.

Thus, in one aspect, the disclosure provides a NOMV vaccine containing a coronavirus receptor binding domain (RBD) modified to be a lipoprotein. In one embodiment, the coronavirus receptor binding domain (RBD) is the SARS-CoV-2 RBD.

In another aspect, the disclosure provides a composition comprising a meningococcal strain containing a gene encoding the SARS-CoV-2 RBD modified to be a lipoprotein, wherein the gene is carried on a plasmid.

In another aspect, the disclosure provides a composition comprising a meningococcal strain having a plasmid-borne gene encoding the SARS-CoV-2 RBD modified to be a lipoprotein with an additional 30-amino-acid segment. In one embodiment, the plasmid comprises a pFP12-RBD plasmid (e.g., SEQ ID NO:8, FIG. 13). In another embodiment, the plasmid comprises a pFP12 SV-40 RBD-2 complete plasmid (e.g., SEQ ID NO:16, FIG. 20). In another embodiment, the plasmid comprises a pFP12 SV40 RBD-2 plasmid (e.g., SEQ ID NO:15, FIG. 7). In another embodiment, the plasmid comprises a pFP12 SV-40 RBD-2 mobC plasmid (e.g., SEQ ID NO:17, FIG. 21). In another embodiment, the plasmid comprises a pUC18-Lpxl1KO-FHbp25RBD-KAN plasmid (e.g., SEQ ID NO:9, FIG. 14). In another embodiment, the plasmid comprises a pGEM-SiaD-GalE-FHbp25RBD-SPC plasmid (e.g., SEQ ID NO:10, FIG. 15). In another embodiment, the plasmid comprises a pBS-FHbpKO-FHbp25RBD-ERM plasmid (e.g., SEQ ID NO:11, FIG. 16).

In other embodiments, one or more plasmids as described herein are introduced into the same bacterial strain. In other embodiments, the meningococcal strain is H44/76 or NZ98/254. In another embodiment, meningococcal strain H44/76 or NZ98/254 does not express porin PorA.

In another aspect, the disclosure provides a meningococcal strain containing a gene encoding the SARS-CoV-2 RBD modified to be a lipoprotein, wherein expression of the gene is driven by a strong promoter sequence that produces high rates of gene transcription in Neisseria meningitidis. In one embodiment, the promoter comprises a sequence set forth herein, such as including, but not limited to, any sequence provided in FIGS. 6-8, 10-21, or SEQ ID NOs:1-17. In another embodiment, the promoter comprises a porin PorA promoter or a derivative thereof, or the promoter of the fumarate and nitrate reductase gene (fnr). In another embodiment, the promoter comprises an EH-NT promoter.

In another aspect, the disclosure provides a meningococcal strain containing a gene encoding the SARS-CoV-2 RBD modified to be a lipoprotein where the gene and promoter are inserted into a locus of the bacterial genome. In one embodiment, the gene and promoter are inserted into the lpxL1 locus to disrupt expression of the acyltransferase gene such that the lipooligosaccharide produced is penta-acylated instead of hexa-acylated. In another embodiment, the gene and promoter are inserted into the siaD-galE locus (also siaA) to disrupt expression of the capsular polysaccharide and sialylation of lipooligosaccharide host antigens. In another embodiment, the gene and promoter are inserted into the fhbp locus (Factor H binding protein). In another embodiment, the gene and promoter are inserted into the porA locus. In one embodiment, inactivation of these genes by homologous recombination is accomplished with plasmids comprising a pUC18-Lpxl1KO-FHbp25RBD-KAN plasmid (e.g., SEQ ID NO:9, FIG. 14), a pGEM-SiaD-GalE-FHbp25RBD-SPC plasmid (e.g., SEQ ID NO:10, FIG. 15), or a pBS-FHbpKO-FHbp25RBD-ERM plasmid (e.g., SEQ ID NO:11, FIG. 16). In other embodiments, one or more plasmids as described herein are introduced into the same bacterial strain. In another embodiment, the SARS-CoV-2 RBD lipoprotein is further modified by adding a 30 amino acid sequence from Nm Factor H binding protein (FHbp) to the lipoprotein signal sequence to facilitate transport of the RBD to the outer surface of the bacteria, ensure proper folding of the RBD and to reduce proteolysis by bacterial proteases. In some embodiments, the 30-amino segment or sequence is from the N-terminus of FHbp, with sequences numbered beginning with lipid-modified Cysteine (Cys) residues on the mature (i.e., processed) protein. In some embodiments, the 30-amino-acid segment or sequence is from a FHbp variant, such as including, but not limited to, ID9. The extended form the RBD lipoprotein also increases the distance between the bacterial surface and the RBD making it more accessible to antigen presenting cells of the human immune system. In other embodiments employing an extension of the lipoprotein signal sequence, in activation of genes by homologous recombination is accomplished with plasmids comprising a pUC18-Lpxl1KO-FHbp55RBD-KAN plasmid (e.g., SEQ ID NO:12, FIG. 17), a pGEM-SiaD-GalE-FHbp55RBD-SPC plasmid (e.g., SEQ ID NO:13, FIG. 18), or a pBS-FHbpKO-FHbp55RBD-ERM plasmid (e.g., SEQ ID NO:14, FIG. 19).

In another embodiment, the disclosure provides an NOMV vaccine containing a coronavirus receptor binding domain (RBD) modified to be a lipoprotein.

In another embodiment, the disclosure provides an NOMV vaccine containing the SARS-CoV-2 RBD modified to be a lipoprotein.

In another embodiment, the disclosure provides a meningococcal strain containing a gene coding for the SARS-CoV-2 RBD modified to be a lipoprotein carried on plasmid (specifically pFP12-RBD, shown in FIG. 13, SEQ ID NO:8).

In another aspect, plasmids maintained in Nm can be incorporated inside NOMV. Thus, NOMV can deliver protein antigens and, at the same time, DNA for the expression of proteins by the cells that take up the NOMV vaccine, to elicit neutralizing antibodies.

The disclosure provides a meningococcal strain containing a plasmid coding for the SARS-CoV-2 RBD with a promoter/enhancer and polyA sequence that provide for expression of the RBD in mammalian cells.

In another aspect, the disclosure provides a NOMV vaccine containing a plasmid coding for the SARS-CoV-2 RBD with a promoter/enhancer and polyA sequence that provide for expression of the RBD in mammalian cells.

In other embodiments the plasmids suitable for incorporation into NOMV for expression of RBD in mammalian cells comprise a pFP12 SV40 RBD-2 plasmid (e.g., SEQ ID NO:15, FIG. 7), a pFP12 SV-40 RBD-2 complete plasmid (e.g., SEQ ID NO:16, FIG. 20), or a pFP12 SV-40 RBD-2 mobC plasmid (e.g., SEQ ID NO:17, FIG. 21).

In another embodiment, the disclosure provides a meningococcal strain containing a gene coding for the SARS-CoV-2 RBD where the meningococcal strain is H44/76 or.

[symbol]

In another embodiment, the disclosure provides a meningococcal strain containing a gene coding for the SARS-CoV-2 RBD modified to be a lipoprotein carried on plasmid where the meningococcal strain is H44/76 which does not express porin PorA.

In another embodiment, the disclosure provides a meningococcal strain containing a gene coding for the SARS-CoV-2 RBD modified to be a lipoprotein where expression of the gene is driven by strong promoter sequence that produces high rates of gene transcription in Neisseria meningitidis. In some embodiments, a specific sequence for a strong promoter as described herein is provided herein, for example in FIGS. 6-8, 10-21, or SEQ ID NOs:1-17. In some embodiments, alternatives for a strong promoter may include, but are not limited to, PorA (see, e.g., U.S. Pat. No. 9,387,239). In some embodiments, a PorA derivative may include, but is not limited to, those described in U.S. Pat. No. 9,260,489 and Canadian Patent No. 2,861,946. In other embodiments, a promoter useful as described herein may include the promoter of the fumarate and nitrate reductase gene (fnr) (Oriente et al., J Bacteriol 192:691-701, 2010). In some embodiments, a promoter useful as described herein may comprise an EH-NT promoter, for example as shown in FIGS. 12-19.

In some embodiments, a promoter useful as described herein may comprise a human EF1 promoter (hEF1), or a hybrid promoter, such as an hEF1-HTLV promoter, which comprises a human Elongation Factor-1α (hEF-1α) core promoter and the 5′ untranslated region of the human T-cell Leukemia virus (HTLV), for example as shown in FIGS. 7 and 20-21. Other strong promoters known or available in the art may be used as described herein, provided they produce high rates of transcription of the gene coding for the SARS-CoV-2 RBD.

In some embodiments, the disclosure provides a meningococcal strain containing a gene coding for the SARS-CoV-2 RBD modified to be a lipoprotein, where the gene and promoter are inserted in the lpxL1 locus to disrupt expression of the acyltransferase gene such that the lipooligosaccharide produced is penta-acylated instead of hexa-acylated.

In some embodiments, the disclosure provides a meningococcal strain containing a gene coding for the SARS-CoV-2 RBD modified to be a lipoprotein, where the gene and promoter are inserted in the siaD-galE locus (also siaA) to disrupt expression of the capsular polysaccharide and sialylation of lipooligosaccharide host antigens.

In some embodiments, the disclosure provides a meningococcal strain containing a gene coding for the SARS-CoV-2 RBD modified to be a lipoprotein, where the gene and promoter are inserted in the fhbp locus (Factor H binding protein).

In some embodiments, the disclosure provides a meningococcal strain containing a gene coding for the SARS-CoV-2 RBD modified to be a lipoprotein, where the gene and promoter are inserted in the porA locus.

In another aspect, the disclosure provides a meningo NOMV vaccine composition comprising a coronavirus receptor binding domain (RBD) modified to be a lipoprotein coccal strain containing a plasmid coding for the SARS-CoV-2 RBD with a promoter/enhancer and polyA sequence that provide for expression of the RBD in mammalian cells.

In another aspect, the disclosure provides a NOMV vaccine containing a plasmid coding for the SARS-CoV-2 RBD with a promoter/enhancer and polyA sequence that provide for expression of the RBD in mammalian cells.

In another aspect, the disclosure provides a method of vaccinating a subject comprising administering an NOMV vaccine composition comprising a coronavirus receptor binding domain (RBD) modified to be a lipoprotein.

These and other embodiments of the disclosure are described in detail below.

### BRIEF DESCRIPTION OF THE SEQUENCES

SEQ ID NO:1—Sequence of C Chain C, Spike glycoprotein receptor binding domain (shown in FIG. 10).

SEQ ID NO:2—Sequence of the signal sequence for the spike glycoprotein, Chain A of SARS-CoV-2 (shown in FIG. 11).

SEQ ID NO:3—Sequence of the mature spike glycoprotein, Chain A of SARS-CoV-2 (shown in FIG. 11).

SEQ ID NO:4—DNA sequence of promoter+RBD (shown in FIG. 12).

SEQ ID NO:5—Protein sequence of the FHbp25-RBD produced in NOMV.

SEQ ID NO:6—Protein sequence of the FHbp55-RBD produced in NOMV.

SEQ ID NO:7—RBD expressed in mammalian cells.

SEQ ID NO:8—DNA sequence of the pFP12 RBD plasmid (FIG. 13).

SEQ ID NO:9—Sequence of pUC18-Lpxl1KO-FHbp25RBD-KAN plasmid (FIG. 14).

SEQ ID NO:10—Sequence of pGEM-SiaD-GalE-FHbp25RBD-SPC plasmid (FIG. 15).

SEQ ID NO:11—Sequence of pBS-FHbpKO-FHbp25RBD-ERM plasmid (FIG. 16).

SEQ ID NO:12—Sequence of pUC18-Lpxl1KO-FHbp55RBD-KAN plasmid (FIG. 17).

SEQ ID NO:13—Sequence of pGEM-SiaD-GalE-FHbp55RBD-SPC plasmid (FIG. 18).

SEQ ID NO:14—Sequence of pBS-FHbpKO-FHbp55RBD-ERM plasmid (FIG. 19).

SEQ ID NO:15—Sequence of pFP12 SV40 RBD-2 plasmid (FIG. 7).

SEQ ID NO:16—Sequence of pFP12 SV-40 RBD-2 complete plasmid (FIG. 20).

SEQ ID NO:17—Sequence of pFP12 SV-40 RBD-2 mobC plasmid (FIG. 21).

SEQ ID NO:18—Sequence of FHbp variant ID9 (FIG. 22).

### BRIEF DESCRIPTION OF THE DRAWINGS

FIG. 1 depicts PCR primers designed to amplify upstream and downstream the constructs inserted in Neisseria meningitidis strain H44/76 carrying the flanking region for the fhbp, siaD-galE, or lpxL1 gene, RBD gene, and the antibiotic resistant cassette.

FIG. 2 shows flow cytometry results of 1 copy (left graph), 2 copies (middle graph), and 3 copies (right graph) of anti-RBD polyclonal antibody (1:1000) binding to the surface of live N. meningitidis bacteria.

FIG. 3 shows a peptide map demonstrating that the RBD was among the most abundant proteins in the NOMV preparation, and peptides representing 65% of the protein including peptides at both N-terminal and C-terminal ends of the protein.

FIG. 4 shows a western blot demonstrating the presence of RBD in the NOMV vaccine.

FIG. 5 shows that mice immunized with NOMV-RBD produced anti-RBD-specific antibodies reactive with both the RBD and the full spike protein (filled symbols) versus mice immunized with aluminum hydroxide adjuvant alone (open symbols).

FIG. 6 shows a mammalian SARS-CoV-2 Spike RBD expression cassette containing the SV40 enhancer, the ubiquitous human EF1α-HTLV composite promoter, and the SV40 polyadenylation (pAn) signal.

FIG. 7 shows the pFP12 SV40 RBD-2 plasmid (SEQ ID NO:15) containing the mammalian SARS-CoV-2 Spike RBD expression cassette (shown in FIG. 6) that was used to transform N. meningitidis.

FIG. 8 depicts the PCR amplification of the upstream and downstream regions of the constructs inserted into N. meningitidis strain H44/76 carrying the flanking region for the siaD-galE, lpxL1 gene, and SV40-RBD gene, and the antibiotic resistant cassette.

FIG. 9 shows PCR amplification of heat-killed cells from different chloramphenicol-resistant bacterial clones (upper panel) and purified NOMV clones (lower panel). Middle panel shows isolation of the plasmid from transformed chloramphenicol-resistant bacterial clones.

FIG. 10 depicts the sequence of the C Chain C, Spike glycoprotein receptor binding domain (SEQ ID NO:1).

FIG. 11 depicts the sequence of the C Chain C, Spike glycoprotein receptor binding domain (SEQ ID NO:1). Boxed highlighted residues indicated by arrows may be involved in glycan binding. Non-boxed highlighted residues contact the ACE2 receptor.

FIG. 12 depicts the DNA sequence of promoter+RBD (SEQ ID NO:4). Shades of tan-highlighted nucleotides indicate the position of 5 overlapping promoter sequences. Cyan-, magenta-, and yellow-highlighted nucleotides indicate restriction sites. Green-highlighted nucleotides indicate the translational start.

FIG. 13 depicts a pFP12-RBD plasmid map (SEQ ID NO:8).

FIG. 14 shows the pUC18-Lpxl1KO-FHbp25RBD-KAN plasmid (SEQ ID NO:9).

FIG. 15 shows the pGEM-SiaD-GalE-FHbp25RBD-SPC plasmid (SEQ ID NO:10).

FIG. 16 shows the pBS-FHbpKO-FHbp25RBD-ERM plasmid (SEQ ID NO:11).

FIG. 17 shows the pUC18-Lpxl1KO-FHbp55RBD-KAN plasmid (SEQ ID NO:12).

FIG. 18 shows the pGEM-SiaD-GalE-FHbp55RBD-SPC plasmid (SEQ ID NO:13).

FIG. 19 shows the pBS-FHbpKO-FHbp55RBD-ERM plasmid (SEQ ID NO:14).

FIG. 20 shows the pFP12 SV-40 RBD-2 complete plasmid (SEQ ID NO:16).

FIG. 21 shows the pFP12 SV-40 RBD-2 mobC plasmid. (SEQ ID NO:17).

FIG. 22 shows the sequence of FHbp variant ID9 (SEQ ID NO:18). The “FHbp55-RBD” sequence is underlined. The “FHbp25-RBD” sequence corresponds to the first 25 amino acids.

### DETAILED DESCRIPTION

### Overview

In one embodiment, the disclosure provides a native outer membrane vesicle (NOMV) vaccine containing a coronavirus receptor binding domain (RBD) modified to be a lipoprotein. In one embodiment, the coronavirus receptor binding domain (RBD) is the SARS-CoV-2 RBD. Other embodiments provide meningococcal strains containing a gene encoding the SARS-CoV-2 RBD modified to be a lipoprotein, wherein the gene is carried on a plasmid, such as a pFP12-RBD plasmid or a pFP12 SV-40 RBD-2 complete plasmid, or a pFP12 plasmid. Some embodiments provide meningococcal strain H44/76 or strain NZ98/254. In some embodiments, meningococcal strain H44/76 or NZ98/254 does not express porin PorA. Other embodiments provide meningococcal strains containing a gene encoding the SARS-CoV-2 RBD modified to be a lipoprotein, wherein expression of the gene is driven by a strong promoter sequence that produces high rates of gene transcription in Neisseria meningitidis, such as a promoter having a sequence set forth herein. In some embodiments, a promoter useful for the present disclosure may be a porin PorA promoter or a derivative thereof. Other embodiments provide a meningococcal strain containing a gene encoding the SARS-CoV-2 RBD modified to be a lipoprotein where the gene and promoter are inserted into a locus of the bacterial genome, such as the lpxL1 locus to disrupt expression of the acyltransferase gene such that the lipooligosaccharide produced is penta-acylated instead of hexa-acylated, or the siaD-galE locus (also siaA) to disrupt expression of the capsular polysaccharide and sialylation of lipooligosaccharide host antigens, or the fhbp locus (Factor H binding protein), or the porA locus.

The present disclosure describes enhanced protective effects of the antibodies against the RBD by (a) over expressing the gene with a novel promoter on a multicopy plasmid and insertion of additional genes in the chromosome to knockout FHbp, capsular polysaccharide and LOS sialylation, (b) displaying it on the surface as a lipoprotein in Nm NOMV, (c) displaying it on the surface as a lipoprotein in Nm NOMV with an additional amino acid segment to facilitate transport to the bacterial surface, proper folding of the RBD, proteolytic stability, and extend the RBD out from the surface of the bacteria to enhance interactions with antigen presenting cells, (d) producing the NOMV in a strain lacking the porin PorA, which is an immunodominant antigen that may, along with capsular polysaccharide, decrease accessibility of the RBD to the immune system, (e) inserting a plasmid into an Nm strain that contains promoter/enhancer and polyA sequences that provide for expression of the RBD in mammalian cells, (f) NOMV vaccine containing a plasmid with promoter/enhancer and polyA sequences that provide for expression of the RBD in mammalian cells.

The meningococcal porin protein PorA is one of the most highly expressed proteins in Nm and elicits high titers of anti-PorA antibodies. However, the PorA promoter that drives expression of the gene is phase variable such that insertion or deletion of bases in a polyG tract during replication can result in increased or decreased expression. The Inventors herein have discovered that the region upstream of the PorA gene in Nm contains 6 potential promoters, of which only one contains the polyG tract. Based on this analysis, the PorA promoter was engineered by removing the sequence containing the polyG tract, thus eliminating the potential for phase variation while retaining the ability to drive high levels of transcription. The engineered promoter construct was used to drive expression of the RBD gene in each of the genes inserted in the chromosome and in the multi-copy plasmid. Promoter-RBD gene constructs were inserted in a region encompassing the siaD and galE to eliminate the production of capsular polysaccharide and sialylation of LOS, fhbp and lpxL1 and in the extrachromosomal plasmid. A variant of Nm strain H44/76 lacking PorA expression was selected to increase accessibility of the RBD and eliminate potential immunologic competition with an immune-dominant antigen of no value in protection against SARS-CoV-2. In some embodiments, Nm strain H44/76 is a useful strain for use with the present disclosure. In another embodiment, Nm strain NZ98/254 is a useful strain for use with the present disclosure.

Proteins displayed on the surface of NOMVs are either integral membrane proteins with one or more transmembrane segments or are modified to be a lipoprotein by the attachment of fatty acids to the amino terminal end of the protein, producing a lipoprotein where the attached fatty acid acts as an anchor to the membrane. Lipoproteins are initially translated as preprolipoproteins, which possess an amino-terminal signal peptide of around 20 amino acids with typical characteristic features of the signal peptides of secreted proteins. A conserved sequence of the signal peptides, referred to as lipobox, having consensus amino acid sequences [LVI][ASTVI][GAS]C, is modified through the covalent attachment of a diacylglycerol moiety to the thiol group on the side chain of the indispensable cysteine residue. This modification is catalyzed by the enzyme lipoprotein diacylglyceryl transferase (Lgt), resulting in a prolipoprotein consisting of a diacylglycerol moiety linked by a thioester bond to the protein. After lipidation, lipoprotein signal peptidase (Lsp or SPase II) is responsible for cleaving the signal sequence of the lipidated prolipoprotein and leaves the cysteine of the lipobox as the new amino-terminal residue. In Gram-negative bacteria, such as Neisseria meningitidis, the cleaved prolipoprotein undergoes an additional modification by attachment of an amide-linked acyl group to the N-terminal cysteine residue by lipoprotein N-acyl transferase (Lnt). The diacylglyceryl group and the amino-terminal acyl group are derived from membrane phospholipids and provide tight anchorage of the lipoprotein to the membrane. To produce the RBD as a surface exposed lipoprotein on NOMV, the Inventors have constructed an RBD coding gene having at the 5′-end sequences coding for an Nm lipoprotein signal sequence.

In addition to the lipoprotein signal sequence, additional amino acid sequences can be added from, for example, Factor H binding protein the enable proper folding and transport of the RBD to the bacterial surface. With proper folding and transport, the RBD is also may be more resistant to proteolysis by bacterial proteases. Also, the additional segment of amino acids serves to extend the RBD out from the bacterial surface where it is more accessible to antigen presenting cells, which can facilitate improved antibody responses to the RBD.

A RBD located within the S-protein and the functional role of the RBD among coronaviruses is conserved. The Inventors have established that the SARS-CoV-2 RBD can be displayed as a lipoprotein on NOMV and the NOMV-RBD vaccine can elicit protective antibodies against SARS-CoV-2. It is anticipated that the RBD domains of other coronaviruses, including the known human pathogens SARS-CoV (72% identical RBD amino acid sequences) and MERS-CoV (17% identical RBD amino acid sequences) can similarly be displayed on NOMV as lipoproteins for use as vaccines to elicit neutralizing antibodies in humans.

The inventors have established that plasmids maintained in Nm can be incorporated into NOMV. As a result, plasmids containing mammalian promoter/enhancer and polyA sequences can be used to express proteins in mammalian cells that take up NOMVs. Thus, NOMV vaccines can provide both protein antigens and protein antigens expressed by the mammalian cells to elicit neutralizing antibodies in humans.

### Definitions

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As used in this specification and the appended claims, the singular forms “a,” “an,” and “the” include plural referents unless the context clearly dictates otherwise. Thus, for example, “an active agent” refers not only to a single active agent, but also to a combination of two or more different active agents, “a dosage form” refers to a combination of dosage forms, as well as to a single dosage form, and the like.

Unless defined otherwise, all technical and scientific terms used herein have the meaning commonly understood by one of ordinary skill in the art to which the disclosure pertains. Specific terminology of particular importance to the description of the present disclosure is defined below.

As used herein, an “adverse event” refers to any untoward medical occurrence associated with the use of a drug or vaccine as described herein in humans, whether or not considered drug related. An AE or suspected adverse reaction may be considered a “serious adverse event” if it results in any of the following outcomes: death, or immediate risk of death, inpatient hospitalization or prolongation of existing hospitalization, persistent or significant incapacity or substantial disruption of the ability to conduct normal life functions, congenital anomaly/birth defect. An adverse event may also be an important medical event that may not result in death, be life-threatening, or require hospitalization, but may jeopardize the patient or subject and may require medical or surgical intervention to prevent one of the above outcomes. In some embodiments, an adverse event refers to an infusion reaction as a result of administration of a drug or vaccine as described herein.

As used herein, “anaphylaxis” refers to a severe, acute onset allergic reaction that may occur over minutes to several hours. Anaphylaxis may involve the skin, mucosal tissue, or both, and may have one or more symptoms including, but not limited to, generalized hives, pruritus (itching), flushing, swelling of the lips, tongue, throat or uvula, shortness of breath, vomiting, lightheadedness, wheezing, hemodynamic instability, and rash or urticaria. In addition, anaphylaxis may be accompanied by at least one of the following: respiratory compromise (e.g., dyspnea, wheeze-bronchospasm, stridor, reduced peak expiratory flow, hypoxemia), and reduced blood pressure (i.e., systolic blood pressure <90 mm Hg or greater than 30% decrease from that person's baseline) or associated symptoms of end-organ failure (e.g., hypotonia [collapse], syncope, incontinence). Anaphylaxis in accordance with the disclosure is defined by the National Institute of Allergy and Infectious Disease/Food Allergy and Anaphylaxis Network (NIAID/FAAN) clinical criteria for diagnosing anaphylaxis.

As used herein, “co-administration” refers to the simultaneous administration of one or more drugs with another. In other embodiments, both drugs are administered at the same time. As described herein elsewhere, co-administration may also refer to any particular time period of administration of either drug, or both drugs. For example, as described herein, a drug may be administered hours, days, weeks, or months before administration of another drug and still be considered to have been co-administered. In some embodiments, co-administration may refer to any time of administration of either drug such that both drugs are present in the body of a patient at the same. In some embodiments, either drug may be administered before or after the other, so long as they are both present within the patient for a sufficient amount of time that the patient received the intended clinical or pharmacological benefits.

As used herein, the terms “effective amount” and “therapeutically effective amount” refer to an amount of an agent, compound, drug, composition or combination which is nontoxic and effective for producing some desired therapeutic effect upon administration to a subject or patient (e.g., a human subject or patient).

As used herein, the term “mammalian cells” refers to any mammal or cells thereof, e.g., a human, in which the SARS-CoV-2 RBD is expressed as described herein.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “reducing” refers to a lowering or lessening, such as reducing symptoms of COVID-19 disease or SARS-CoV-2 infection. In some embodiments, administration of a vaccine as described herein, such as a NOMV vaccine, may result in “reduced” or lessened symptoms in the patient compared to a patient not been administered such a vaccine. “Reducing” may also refer to a reduction in disease symptoms as a result of a treatment as described herein, either alone, or co-administered with another drug.

As used herein, “subject” or “individual” or “patient” refers to any patient for whom or which therapy is desired, and generally refers to the recipient of the therapy.

The terms “treating” and “treatment” as used herein refer to reduction in severity and/or frequency of symptoms, elimination of symptoms and/or underlying cause, and improvement or remediation of damage. In certain aspects, the term “treating” and “treatment” as used herein refer to the prevention of the occurrence of symptoms. In other aspects, the term “treating” and “treatment” as used herein refer to the prevention of the underlying cause of symptoms associated with a disease, such as COVID-19 disease. The phrase “administering to a patient” refers to the process of introducing a composition, vaccine, or dosage form into the patient via an art-recognized means of introduction.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1

### Replacing fhbp, siaD-galE, and lpxL1 Genes by Insertion of a Gene Coding for the RBD with a Meningococcal Lipoprotein Signal Sequence

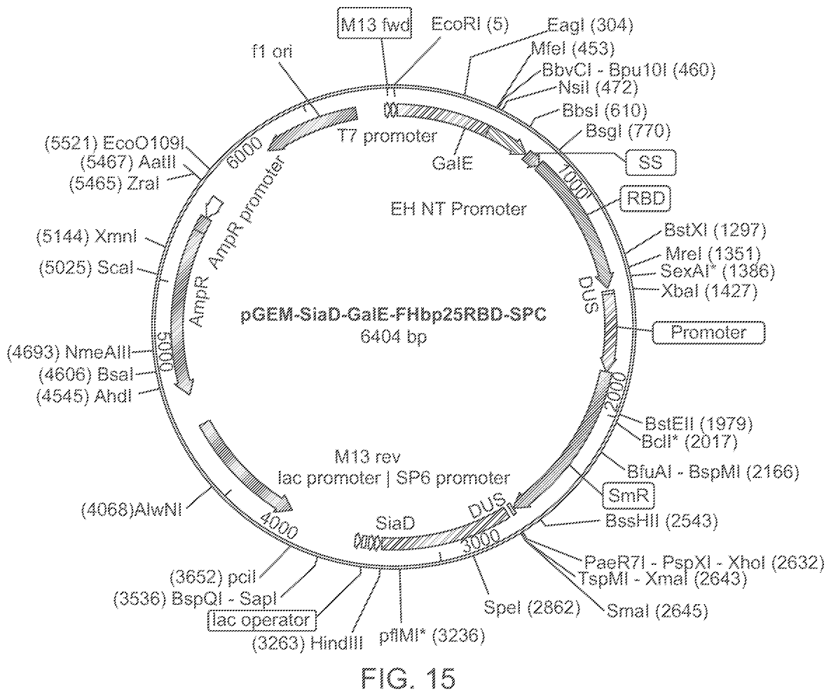

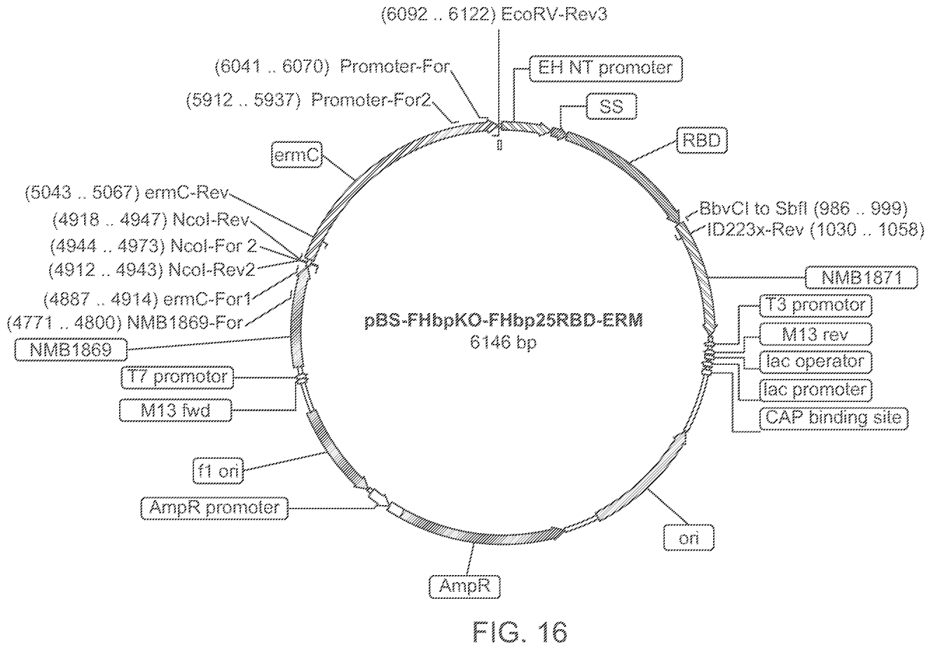

The DNA segment containing a promoter (EH-NT), the Neisserial Factor H binding protein (FHbp) lipoprotein leader sequence from FHbp ID9, and the RBD sequence (referred to as the EH-NT-ID9 SS-RBD sequence) was cloned into vectors for knocking out lpxL1 (pUC18-lpxL1KO-RBD-KAN plasmid), galE-siaD (pGEM-SiaD-GalE-FHbp25RBD-SPC plasmid, SEQ ID NO:10, FIG. 15), and the FHbp gene NMB1870 (pBS-FHbpKO-FHbp25RBD-ERM plasmid, SEQ ID NO:11, FIG. 16). Bacteria were mixed with 3 μg of the plasmid, plated onto a tryptic soy broth (TSB) agar plate, and incubated for 6 hrs at 37° C. Serial dilutions of the bacteria were re-cultured onto TSB agar plates containing kanamycin (50 μg/mL), spectinomycin (50 μg/mL), and erythromycin (10 μg/mL), respectively. The culture plates were incubated overnight at 37° C., and the resulting colonies were screened for RBD expression and for the lack of FHbp, capsular polysaccharide, and LpxL1 by a flow cytometry assay using specific antibodies and by PCR using heat-killed cells. Positive individual colonies were frozen in 10% skim milk (wt/vol) and 15% glycerol, and stored at −80° C.

### Example 2

### Characterization of Mutant of Neisseria meningitidis (Nm) Strain H44/76 Containing 3 Chromosomal Copies Coding for the RBD and a Multi-Copy Plasmid Coding for the RBD, Each with an Nm Lipoprotein Signal Sequence (PCR, FACS)

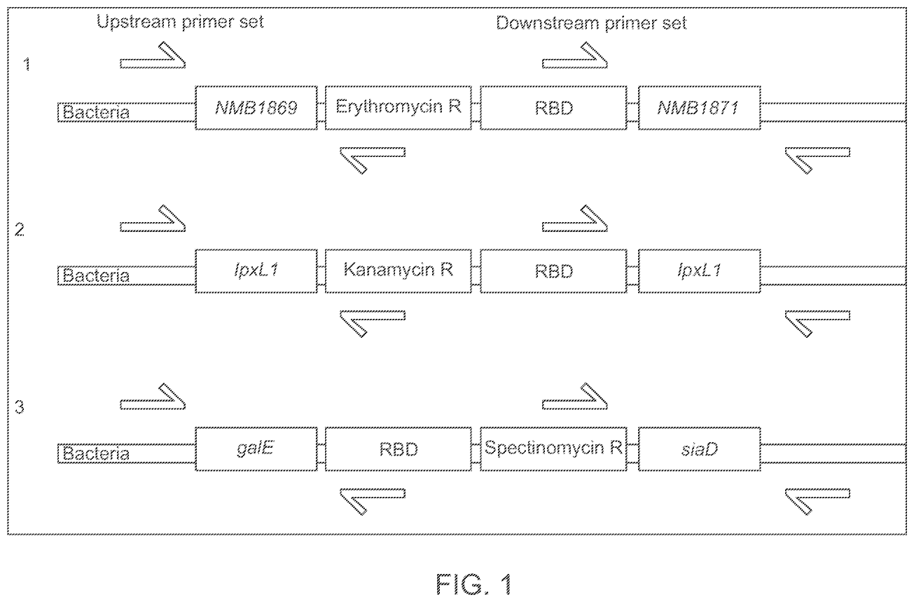

PCR: PCR primers were designed in order to amplify upstream and downstream the constructs inserted in Neisseria meningitidis strain H44/76 carrying the flanking region for the fhbp, siaD-galE, or lpxL1 genes, the RBD gene, and the antibiotic resistant cassette (see FIG. 1). PCR was performed on heat-killed cells. The heat killed cells from the wild-type H44/76 were used as negative control.

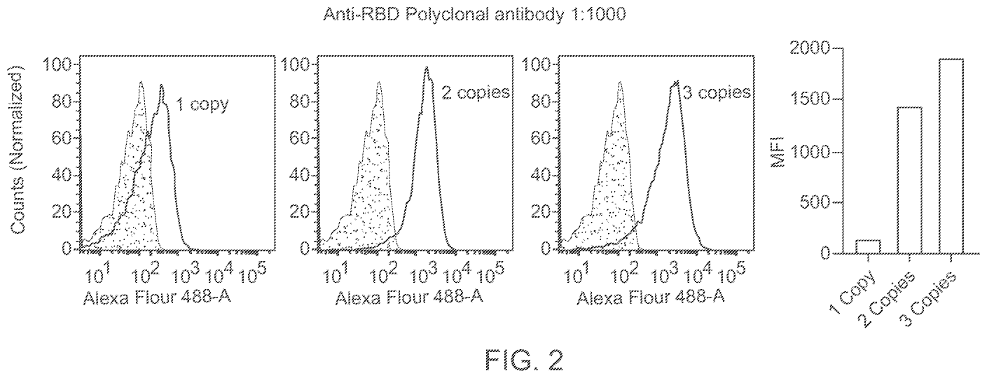

Flow cytometry: Binding of anti-SARS-COV-2 Spike RBD rabbit polyclonal antibody (myBiosource Cat #MBS2563840) to the surface of live N. meningitidis bacteria was measured by flow cytometry as described previously (Giuntini et al., Clin Vaccine Immunol 23:698-706, 2016). Briefly, bacteria were grown in chemically defined medium up to an OD_{620 nm} of 0.6-0.7. To measure anti-RBD antibody binding, a fixed concentration of anti-RBD antibodies or, as a negative control, 10 μg/mL of an irrelevant antibody, was incubated with 10⁷ bacteria/mL. Bound antibody was detected using AlexaFluor 488-conjugated goat anti-rabbit IgG [F(ab′)2 (H+L)] secondary antibody (Jackson Immuno Research Laboratories). As shown in FIG. 2, each additional copy introduced into the bacteria resulted in increased fluorescence detected by flow cytometry.

### Example 3

### Preparation and Characterization of NOMV Vaccine Containing the RBD (Western Blot, LC/MS/MS)

NOMV Preparation:

NOMVs were obtained from medium inoculated with bacteria to an OD_{620 nm} of 0.15-0.2 from overnight colonies of bacteria on TSB agar plates. The culture was incubated at 37° C. in 5% CO₂, and the volume of medium was increased before the culture reached stationary phase (OD_{620 nm} of 0.6-0.7, usually every 1.5 hours). When the final volume was reached, the culture was left to grow for an additional 15 hours in a shake flask with vented enclosure. The bacteria were then centrifuged (10,000×g, 20 minutes), the supernatant filtered through a glass fiber filter to remove debris, then sterile filtered (0.22 μm filter), and concentrated by ultrafiltration (100 k cutoff filter, Amicon) and benzonase added (1000 U/L). Benzonase treatment was continued for at least 1 hr at ambient temperature. The concentrated filtrate was centrifuged (202,601×g, 1.5 hrs, 4° C.) to collect the NOMV. The NOMV were suspended in 10 mM Tris·HCl, pH 7.4, 3% (w/v) sucrose, centrifuged again as described in the previous step, and finally suspended in the Tris/sucrose solution to a concentration between 1 to 3 mg/mL protein as determined by DC Protein Assay (Bio-Rad). The NOMV preparation was stored at −70° C. until used.

Liquid Chromatography Mass Spectrometry (LC-MS/MS) Peptide Mapping:

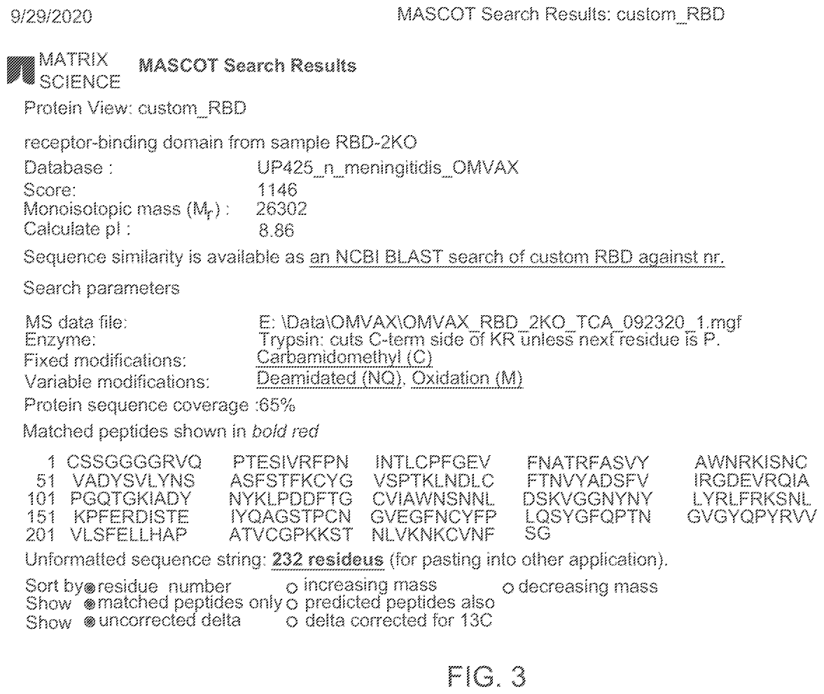

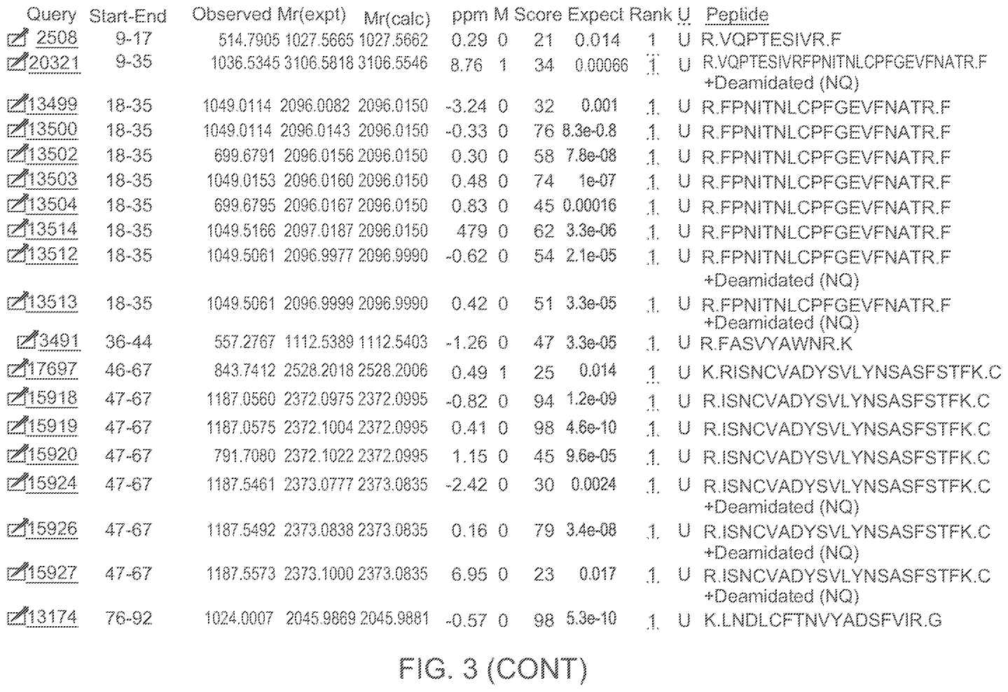

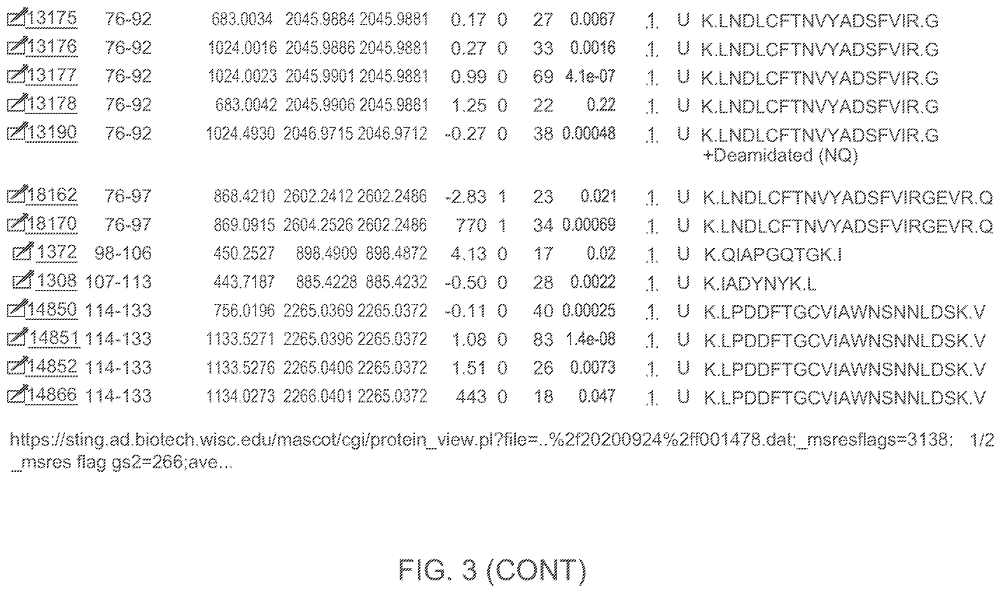

To confirm that the RBD with the FHbp ID9 lipoprotein signal sequence was present in NOMV-RBD, the NOMV were precipitated from solution (ProteoExtract® Protein Precipitation Kit, EMDMillipore), reconstituted in 6M urea/5 mM dithiothreitol, the cysteine residues modified with iodoacetamide, and digested with a mixture of Lys-C/trypsin (Promega). The resulting peptides were purified on MacroSpin columns (Nest Group, Inc.) and submitted for peptide mapping at the University of Wisconsin Biotechnology Center Mass Spectrometry Facility (Madison, Wis.). The resulting peptide map showed that the RBD was among the most abundant proteins in the NOMV preparation and peptides representing 65% of the protein including peptides at both N-terminal and C-terminal ends of the protein (FIG. 3).

Western Blot:

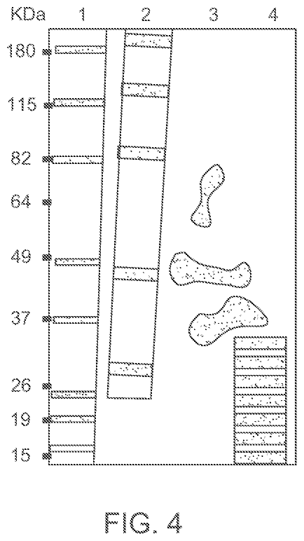

To show the presence of RBD in the NOMV vaccine containing RBD (NOMV-RBD), preparations of purified recombinant RBD (Sino Biological), NOMV without RBD, and NOMV-RBD were resolved on 4%-12% SDS-PAGE gels (NuPAGE, Thermo Fisher Scientific) and transferred to a PVDF membrane (Immobilon®-FL, Millipore) using a semi-dry transfer cell (Trans-Blot® SD, Bio-Rad, Hercules, Calif.) for western blotting. The PVDF membrane was blocked overnight with 5% dry whole milk in phosphate buffered saline (PBS) buffer, then the membrane was stained in blocking buffer for 2 hours at ambient temperature with anti-SARS-COV-2 Spike RBD rabbit polyclonal antibody (myBiosource Cat #MBS2563840). After washing three times with PBS buffer, the bound antibodies were detected with IRDye® 685CW-conjugated donkey anti-goat IgG (H+L) secondary antibody (LI-COR, Lincoln, Nebr.) Images of gels and blots were recorded on an Odyssey® Fc Imaging System (LI-COR). As shown in FIG. 4, the recombinant RBD runs as a mixture of trimers, dimers, and monomers (lane 2). The presence of lipidated RBD in NOMV is shown by the upper arrow in lane 4, and the non-lipidated form indicated the lower arrow. Lane 3 contains NOMV not containing the RBD.

Immunization:

Mice were immunized with two doses, three weeks apart, of RBD-NOMV at 10 μg/dose formulated with 100 μg/dose of Alhydrogel® (InvivoGen). As positive and negative controls respectively, mice were immunized with recombinant RBD (Sino Biological Cat. #40592-V08H) 10 μg/dose formulated with 100 μg/dose of Alhydrogel® or 100 μg/dose of Alhydrogel® only.

Blood samples were collected 5 days post-dose 2 and tested for anti-RBD antibodies by ELISA. Plates were coated with 2 μg/mL of recombinant RBD (Sino Biological Cat. #40592-V08H) or recombinant Spike S1+S2 (Sino Biological Cat #40589-V08B1).

### Example 4

### NOMV-RBD Vaccine Elicits Antibodies to Recombinant RBD by ELISA

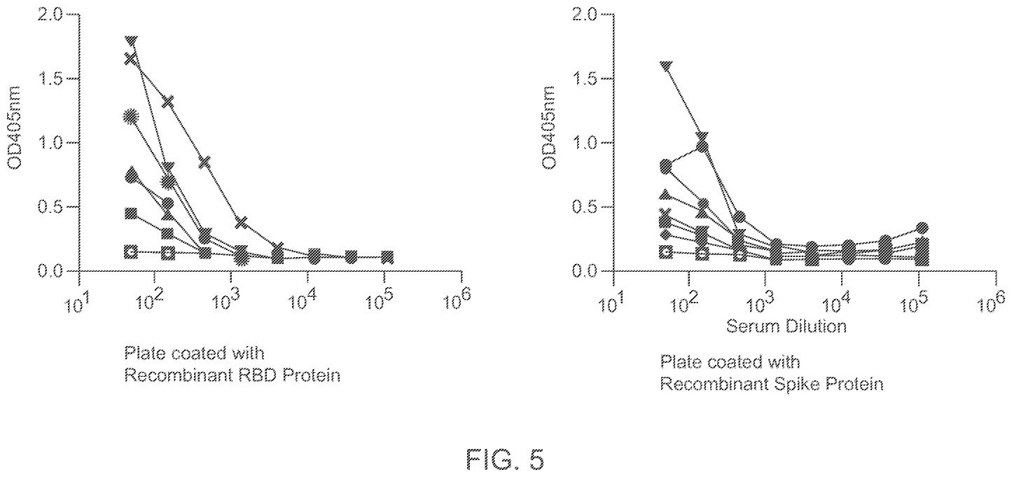

Groups of 7 outbred CD1 mice were immunized with a 10 μg dose of NOMV-RBD. Blood samples were obtained after 3 weeks. Binding activity of polyclonal antibodies raised in mice immunized with NOMV-RBD vaccine was determined by ELISA performed as follows. 96-well plates (Nunc) were coated overnight at 4° C. with 2 μg/mL recombinant SARS-CoV-2 receptor binding domain (RBD) or full spike protein (Sino Biological). Plates were blocked with 1% BSA (weight/volume)+0.05% Tween 20 in phosphate buffered saline (PBS). Sera from immunized mice were diluted in PBS+0.1% Tween 20 and added to plates overnight. The bound antibodies were detected with alkaline phosphatase-conjugated goat anti-mouse IgG F(ab′)2 (H+L) (Jackson Immuno Research Laboratories) (1:3,000 dilution) for 1 hour and developed using p-nitrophenyl phosphate (Thermo Fisher Scientific). Absorbance at an OD_{405 nm} was measured on an Emax precision plate reader (Molecular Devices). As shown in FIG. 5 (right panel), all 7 mice produced anti-RBD-specific antibodies reactive with both the RBD and full spike protein (filled symbols). None of the mice immunized with aluminum hydroxide adjuvant alone (open symbols) had antibodies to the RBD (right panel).

### Example 5

### Construction of a Plasmid Carried Inside NOMV for Expression of the RBD in Mammalian Cells

In this example, it is demonstrated that a plasmid containing the RBD with a signal sequence, promoter/enhancer sequences, and polyA tail is taken up inside NOMV. NOMV-RBD produced from a stain with three copies of the RBD and the plasmid has the potential to deliver a protein antigen to stimulate an antibody response, plus the RBD gene inside cells where the gene can be expressed, further stimulating protective antibody responses.

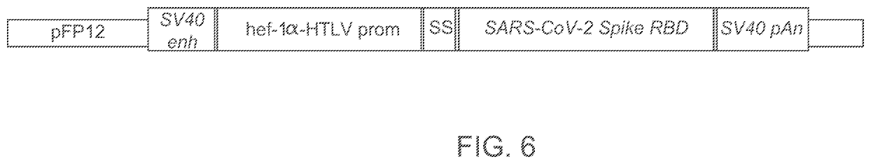

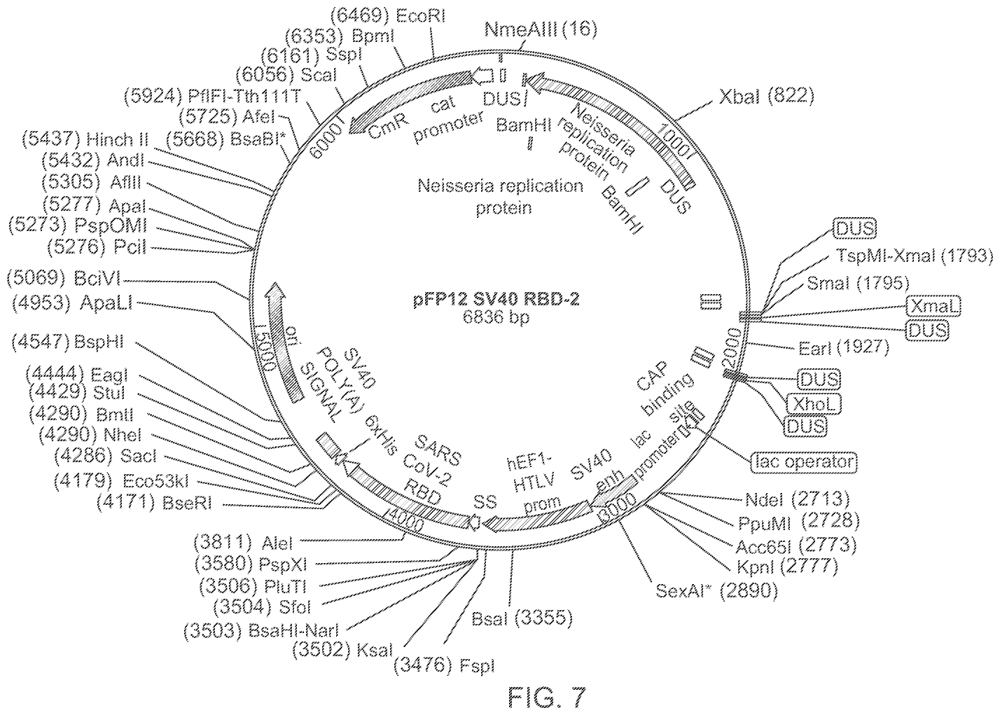

The multi copy plasmid was engineered to include a mammalian expression cassette comprised of the SV40 enhancer, the ubiquitous human EF1α-HTLV composite promoter, and the SV40 polyadenylation (pAn) signal (FIG. 6). A map of a resulting plasmid used to transform Nm strain is shown in FIG. 7.

### Example 6

### Transformation of N. meningitidis

The H44/76 strain, in which the siaD-galE, and lpxL1 genes were inactivated (H44/76ΔFHbp ΔCapsule ΔlpxL1), was made by homologous recombination by transformation with plasmids pGEM-SiaD/GalEKO-SPC using spectinomycin selection (50 μg/mL) and pUC18-lpxL1KO-KAN using kanamycin selection (50 μg/mL) and pFP12-SV40-RBD-CAT using chloramphenicol (5 μg/mL). Transformations starting from the wild-type strain were carried in this order:

The capsule genes were knocked out (pGEM-SiaD-GalE-FHbp25RBD-SPC plasmid, SEQ ID NO:10, FIG. 15)

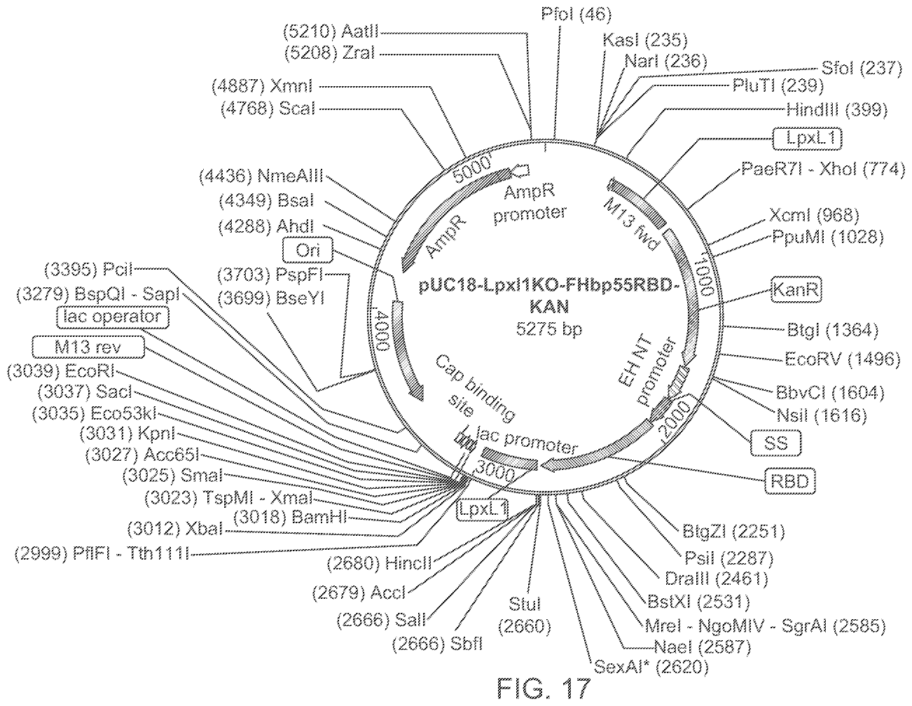

The lpxL1 gene was knocked out (pUC18-Lpxl1KO-FHbp55RBD-KAN plasmid, SEQ ID NO:12, FIG. 17)

Mammalian expression cassette for RBD (pFP12 SV40 RBD-2 plasmid, SEQ ID NO:15, FIG. 7)

Ten to 15 colonies of the H44/76 strain were selected from a TSB (Tryptic Soy Broth, non-animal origin) agar plate that had been grown overnight. The colonies of bacteria were mixed with 3 μg of the plasmid, plated onto a TSB agar plate, and incubated for 6 hrs at 37° C. Serial dilutions of the bacteria were re-cultured onto TSB agar plates containing antibiotic for selection. The culture plates were incubated overnight at 37° C., and the colonies were screened for lack of Capsule and lpxL1 by a flow cytometry assay using specific antibodies and by PCR using heat killed cells. Positive individual colonies were frozen in 10% skim milk (wt/vol) and 15% glycerol, and stored at −80° C. PCR using heat killed cells was done to detect the presence of the SV40-RBD cassette. Chloramphenicol resistant colonies were grown overnight in Frantz and chloramphenicol, and plasmid DNA was isolated the next day and ran on as SDS-PAGE gel to confirm the presence of the multi copy plasmid.

### Example 7

### PCR on Heat-Killed Cells

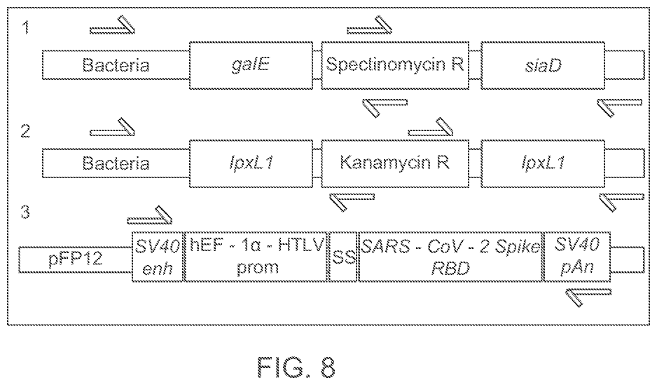

PCR primers were design in order to amplify upstream and downstream the constructs inserted in N. meningitidis strain H44/76 carrying the flanking region for the siaD-galE, or lpxL1 gene, SV40-RBD gene, and the antibiotic resistant cassette (see FIG. 8). PCR was performed on heat-killed cells. The heat-killed cells from the wild-type H44/76 were used as negative control.

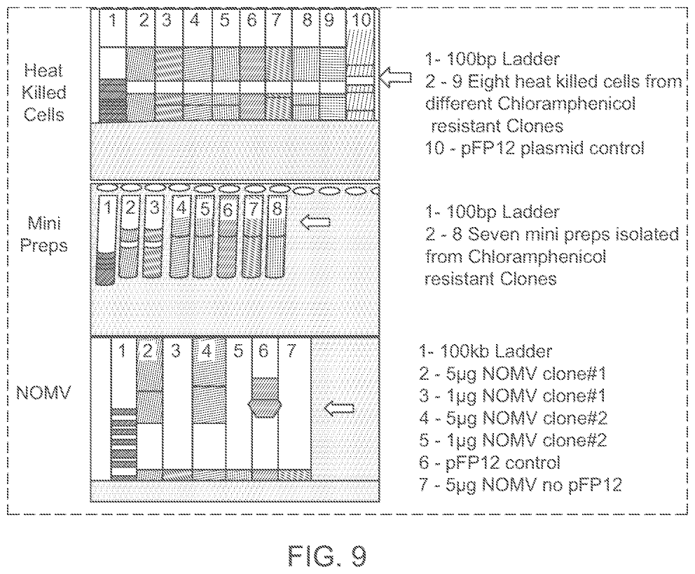

As shown in FIG. 9, the presence of pFP12 plasmid in transformed bacteria can be confirmed by PCR on heat killed cells (upper panel) and on purified NOMV (lower panel). The plasmid can be isolated from transformed bacteria and detected on a DNA gel (middle panel). Arrows indicate size of expected bands.

### Example 8

### Expression of Plasmid-Encoded Nucleic Acid in Mammalian Cells

In some embodiments, the present disclosure provides a meningococcal strain or a NOMV vaccine containing a plasmid coding for the SARS-CoV-2 RBD with a promoter/enhancer and polyA sequence that provide for expression of the RBD in mammalian cells. As described herein, NOMV have the SARS-CoV-2 RBD present on the outside of the vesicles, which will bind to ACE2 receptors on a cell, such as a human or other mammalian cell. Upon binding, those cells will take up the NOMV particle as a whole, which, in addition to SARS-CoV-2 RBD presented on the NOMV surface, also contains nucleic acid coding for the SARS-CoV-2 RBD. Those cells, along with other types of antigen-presenting cells (APCs) in the subject, will bind to the NOMV and will express the SARS-CoV-2 RBD, similar to mRNA or adenoviral-type vaccines, which deliver nucleic acid to the cell, resulting in expression of the protein. Therefore, the expressed protein will be expressed and secreted by those cells, eliciting an antibody response in the subject. Thus, in some embodiments, an antigen (e.g., SARS-CoV-2 RBD) is provided as a protein on the surface of NOMV, while also being provided as a nucleic acid that results in expression and secretion from the cells that have taken up the NOMV.

### Example 9

### Administration of NOMV Vaccine Composition

The present disclosure provides a method of vaccinating a subject comprising administering a NOMV vaccine composition comprising a coronavirus receptor binding domain (RBD) modified to be a lipoprotein. Administration of a vaccine composition may comprise administering (a) NOMV having SARS-CoV-2 RBD displayed on the surface, or (b) NOMV having SARS-CoV-2 RBD displayed on the surface in addition to expressing a plasmid encoding the SARS-CoV-2 RBD as described herein.

A NOMV vaccine preparation as described herein comprises from about 6 μg to about 60 μg of NOMV alone or adsorbed to adjuvant, such as about 0.25 mg to about 0.6 mg of Al³⁺ as aluminum hydroxide (Alhydrogel®, Brentag) in 10 mM Tris, pH 7.5, buffer containing 0.9% NaCl, and 3% (weight/volume) sucrose. The amount of NOMV and adjuvant may be altered prior to administration as deemed appropriate by a clinician or physician. Alternatively, the NOMV may be formulated with other vaccine adjuvants suitable for use in humans, such as other aluminum salts (e.g., aluminum hydroxide, aluminum hydroxyphosphate), QS-21, mixtures of aluminum salts with QS21, CpG 1018® (Dynavax), or oil in water adjuvants [e.g., MF59® (GSK), AS03® (GSK)]. Subjects are given up to 3 intramuscular injections of the NOMV vaccine in a 0.5 mL volume at intervals of at least 2 months. Protective antibody responses are measured by ELISA as described in Example 4, surrogate viral neutralization assay (e.g., SARS-CoV-2 Surrogate Virus Neutralization Test (sVNT) Kit (ProteoGenix); cPass™ SARS-CoV-2 Neutralization Antibody Detection Kit (Genescript)), and reduction in viral load by PCR test in subjects exposed to natural infection.

What is claimed is:

1. A NOMV vaccine composition comprising a coronavirus receptor binding domain (RBD) modified to be a lipoprotein.

2. The NOMV vaccine composition of claim 1, wherein the coronavirus RBD is from SARS-CoV-2.

3. The composition of claim 2, wherein the composition comprises a meningococcal strain having a plasmid-borne gene encoding the SARS-CoV-2 RBD modified to be a lipoprotein.

4. The composition of claim 3, wherein the plasmid-borne gene encoding the SARS-CoV-2 RBD modified to be a lipoprotein comprises an additional 30-amino-acid segment.

5. The composition of claim 4, wherein the 30-amino-acid segment is from the amino terminus of Neisseria meningitidis Factor H binding protein (FHbp).

6. The composition of claim 5, wherein the 30-amino-acid segment is attached to a lipoprotein signal sequence to facilitate transport of the RBD to the outer surface of the meningococcal strain.

7. The composition of claim 3, wherein the plasmid comprises a pFP12-RBD plasmid.

8. The composition of claim 2, wherein the plasmid comprises a pFP12 SV-40 RBD-2 complete plasmid.

9. The composition of claim 2, wherein the plasmid comprises a pFP12 SV40 RBD-2 plasmid.

10. The composition of claim 2, wherein the plasmid comprises a pFP12 SV-40 RBD-2 mobC plasmid.

11. The composition of claim 3, wherein the plasmid comprises a pUC18-Lpxl1KO-FHbp25RBD-KAN plasmid.

12. The composition of claim 3, wherein the plasmid comprises a pGEM-SiaD-GalE-FHbp25RBD-SPC plasmid.

13. The composition of claim 3, wherein the plasmid comprises a pBS-FHbpKO-FHbp25RBD-ERM plasmid.

14. The composition of claims 3-13, wherein the meningococcal strain is H44/76 or NZ98/254.

15. The composition of claim 14, wherein the meningococcal strain does not express porin A (PorA).

16. A composition comprising a meningococcal strain having a gene encoding the SARS-CoV-2 RBD modified to be a lipoprotein, wherein expression of the gene is driven by a promoter sequence that produces high rates of gene transcription in Neisseria meningitidis.

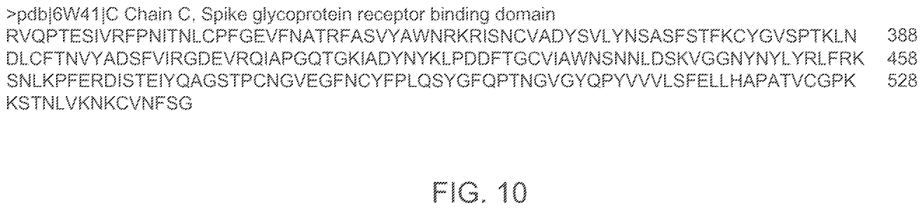

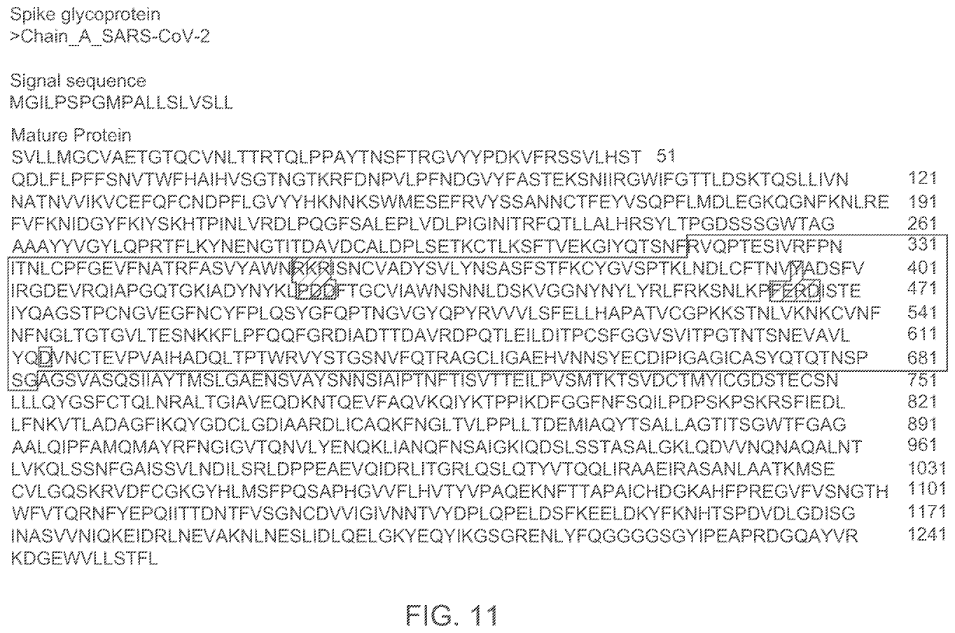

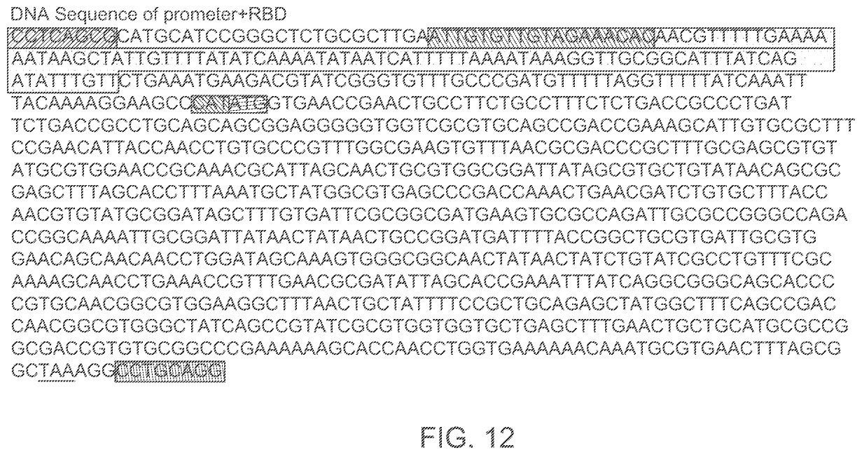

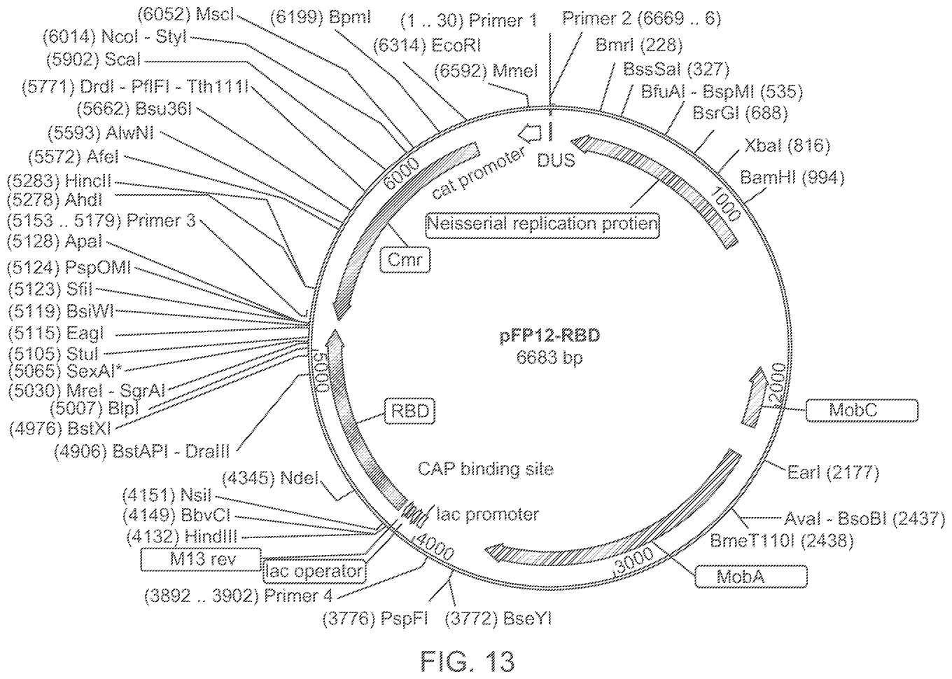

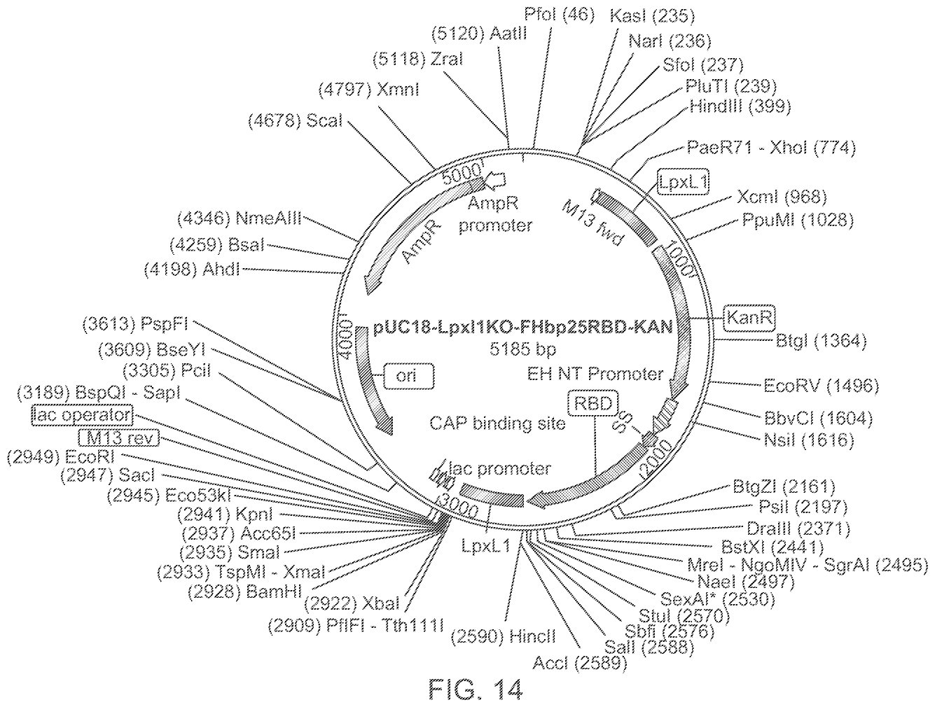

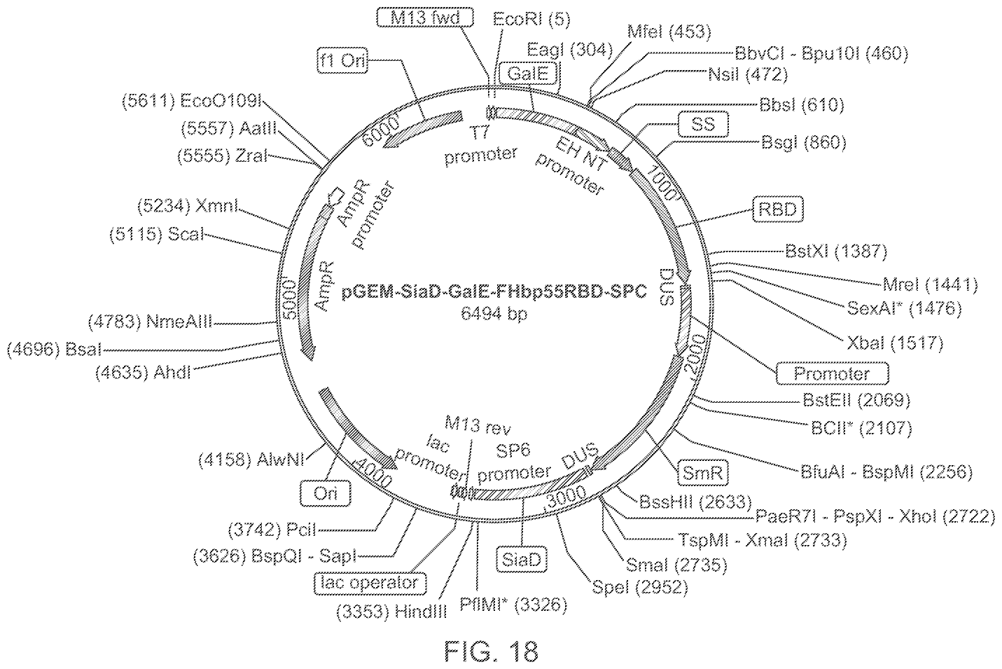

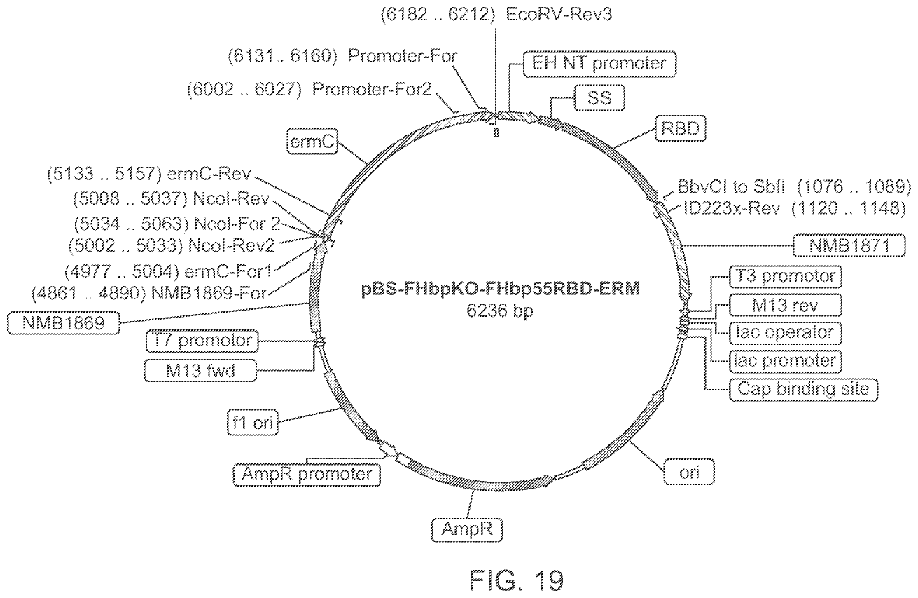

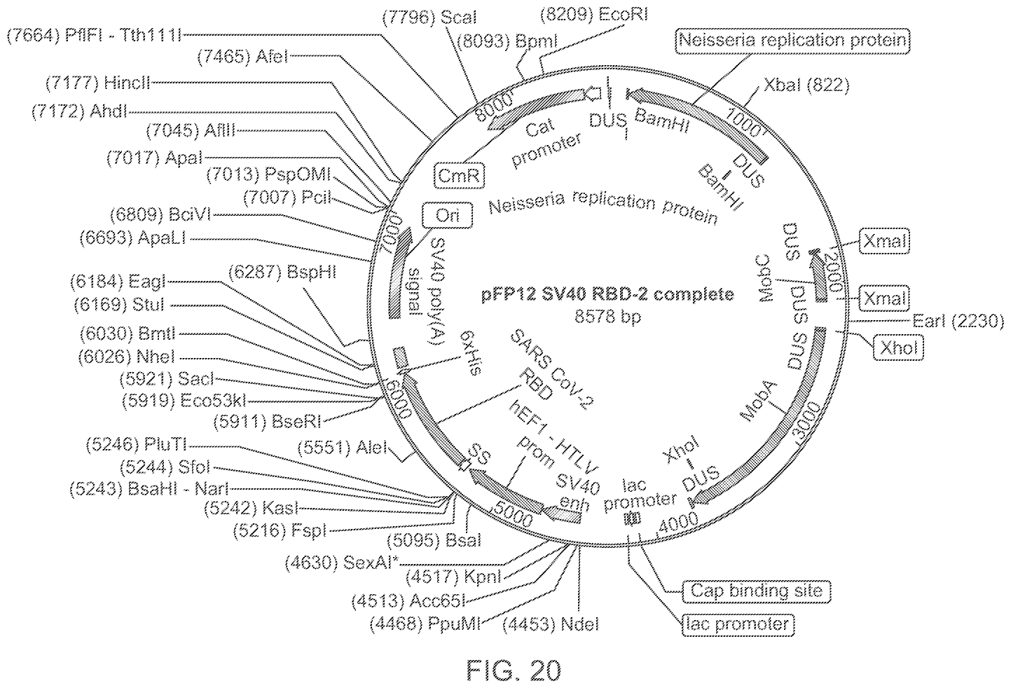

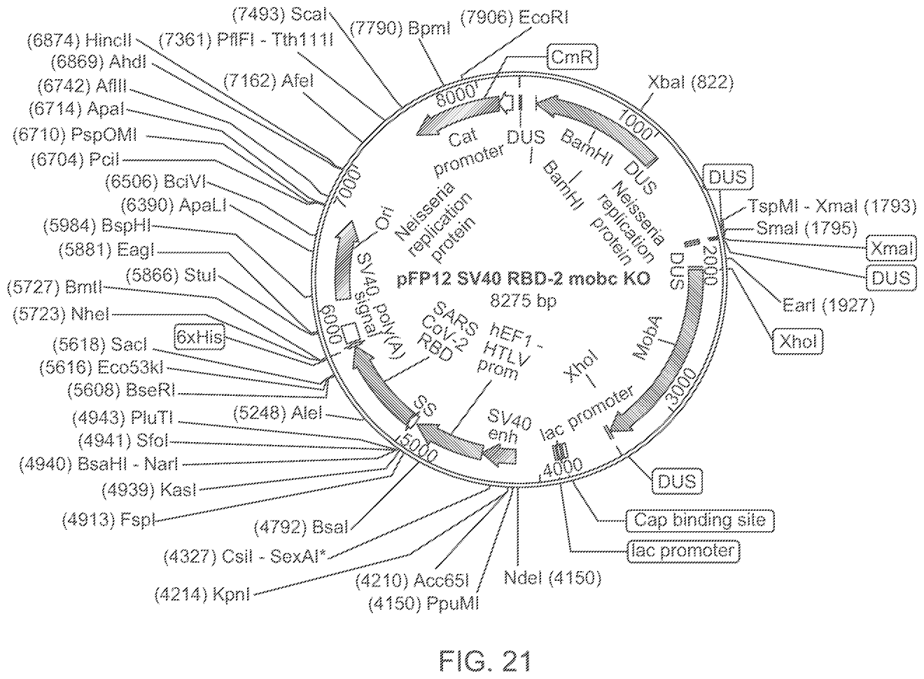

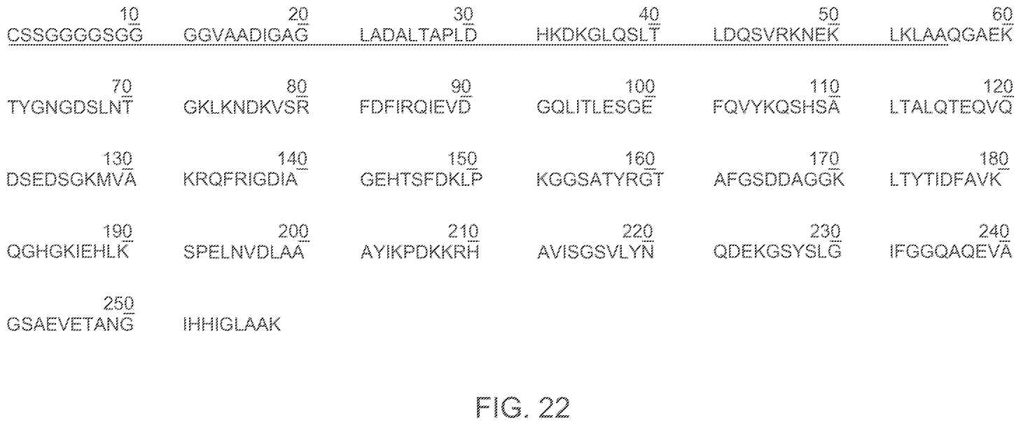

17. The composition of claim 16, wherein the promoter comprises a sequence set forth in FIGS. 6-8, 10-21, or SEQ ID NOs:1-17.

18. The composition of claim 16, wherein the promoter comprises the promoter of porin A (PorA) or a derivative thereof, or the promoter of the fumarate and nitrate reductase gene (fnr).

19. The composition of claim 16, wherein the promoter comprises an EH-NT promoter.

20. The composition of claim 16, wherein the gene and promoter are inserted into a locus of the bacterial genome.

21. The composition of claim 20, wherein the gene and promoter are inserted into the lpxL1 locus, and wherein expression of the acyltransferase gene is disrupted such that the lipooligosaccharide produced is penta-acylated and not hexa-acylated.

22. The composition of claim 20, wherein the gene and promoter are inserted into the siaD-galE locus (also siaA), and wherein expression of the capsular polysaccharide and sialylation of lipooligosaccharide host antigens are disrupted.

23. The composition of claim 20, wherein the gene and promoter are inserted into the fhbp locus (Factor H binding protein).

24. The composition of claim 20, wherein the gene and promoter are inserted into the porA locus.

25. A meningococcal strain containing a plasmid coding for the SARS-CoV-2 RBD with a promoter/enhancer and polyA sequence that provide for expression of the RBD in mammalian cells.

26. A NOMV vaccine containing a plasmid coding for the SARS-CoV-2 RBD with a promoter/enhancer and polyA sequence that provide for expression of the RBD in mammalian cells.

27. A method of vaccinating a subject comprising administering the composition of claim 1.
